SEQUENCE LISTING (40 sequences)

SEQ ID NO: 1 - protein, 253 aa, Saponaria officinalis
VTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPK

SEQ ID NO: 2 - protein, 11 aa, synthetic construct
ACDCRGDCFCG

SEQ ID NO: 3 - protein, 25 aa, synthetic construct
FETLRGDERILSILRHQNLLKELQD

SEQ ID NO: 4 - protein, 6 aa, synthetic construct
CNGRCG

SEQ ID NO: 5 - protein, 7 aa, synthetic construct
GGSSRSS

SEQ ID NO: 6 - protein, 280 aa, synthetic construct
ACDCRGDCFCGGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKVDKHHHHHH

SEQ ID NO: 7 - protein, 275 aa, synthetic construct
CNGRCGGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKVDKHHHHHH

SEQ ID NO: 8 - protein, 294 aa, synthetic construct
FETLRGDERILSILRHQNLLKELQDGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKVDKHHHHHH

SEQ ID NO: 9 - DNA, 840 bp, synthetic construct
  source 1..840: mol_type = other DNA
gcttgtgattgtaggggagattgtttttgtggagggggatccagtagatcaagcgtgaccagcatcaccctggacctggttaacccgaccgcgggccagtacagcagctttgtggacaagattcgtaacaacgttaaggacccgaacctgaaatatggcggtaccgatatcgcggtgattggtccgccgagcaaggagaaattcctgcgtatcaactttcaaagcagccgtggcaccgttagcctgggtctgaagcgtgacaacctgtacgtggttgcgtatctggcgatggataacaccaacgtgaaccgtgcgtactatttcaaaagcgagattaccagcgcggaactgaccgcgctgtttccggaagcgaccaccgcgaaccagaaggcgctggagtacaccgaagactatcaaagcatcgagaaaaacgcgcagattacccaaggtgacaagagccgtaaagaactgggcctgggtatcgatctgctgctgaccttcatggaggcggttaacaagaaagcgcgtgtggttaagaacgaagcgcgtttcctgctgatcgcgattcagatgaccgcggaagtggcgcgttttcgttacatccaaaacctggttaccaagaacttcccgaacaaatttgacagcgataacaaagtgattcagttcgaagttagctggcgtaaaatcagcaccgcgatttacggcgatgcgaagaacggtgtgtttaacaaagactatgatttcggctttggcaaagtgcgtcaggttaaagacctgcaaatgggcctgctgatgtatctgggcaagccgaaagtcgacaaacaccaccaccaccaccac

SEQ ID NO: 10 - DNA, 825 bp, synthetic construct
  source 1..825: mol_type = other DNA
tgtaatggaaggtgtggagggggatccagtagatcaagtgtaacatcaataaccctggacctggttaacccgaccgcgggccagtacagcagcttcgtggacaagattcgtaacaacgttaaggacccgaacctgaaatatggcggtaccgatatcgcggtgattggtccgccgagcaaggagaaattcctgcgtatcaactttcaaagcagccgtggcaccgttagcctgggtctgaagcgtgacaacctgtacgtggttgcgtatctggcgatggataacaccaacgtgaaccgtgcgtactatttcaaaagcgagattaccagcgcggaactgaccgcgctgtttccggaagcgaccaccgcgaaccagaaggcgctggagtacaccgaagactatcaaagcatcgagaaaaacgcgcagattacccaaggtgacaagagccgtaaagaactgggcctgggtatcgatctgctgctgacctttatggaggcggttaacaagaaagcgcgtgtggttaagaacgaagcgcgtttcctgctgatcgcgattcagatgaccgcggaagtggcgcgttttcgttacatccaaaacctggttaccaagaacttcccgaacaaatttgacagcgataacaaagtgattcagttcgaagttagctggcgtaaaatcagcaccgcgatttacggcgatgcgaagaacggtgtgtttaacaaagactatgatttcggctttggcaaagtgcgtcaggttaaagacctgcaaatgggcctgctgatgtatctgggcaagccgaaagtcgacaaacaccaccaccaccaccac

SEQ ID NO: 11 - DNA, 882 bp, synthetic construct
  source 1..882: mol_type = other DNA
tttgaaacactaaggggagatgaaagaatattatcaatactaaggcaccaaaacctgctgaaggagctgcaagacggtggatccagccgtagcagcgtgaccagcatcaccctggatctggttaacccgaccgcgggccagtacagcagctttgtggacaaaattcgtaacaacgttaaggacccgaacctgaaatatggtggcaccgatatcgcggtgattggtccgccgagcaaggagaaattcctgcgtatcaactttcaaagcagccgtggtaccgttagcctgggcctgaagcgtgacaacctgtacgtggttgcgtatctggcgatggataacaccaacgtgaaccgtgcgtactatttcaaaagcgagattaccagcgcggaactgaccgcgctgtttccggaagcgaccaccgcgaaccagaaggcgctggagtacaccgaagactatcaaagcatcgagaaaaacgcgcagattacccaaggcgacaagagccgtaaagaactgggtctgggcatcgatctgctgctgaccttcatggaggcggttaacaagaaagcgcgtgtggttaagaacgaagcgcgtttcctgctgatcgcgattcagatgaccgcggaagtggcgcgttttcgttacatccaaaacctggttaccaagaacttcccgaacaaatttgacagcgataacaaagtgattcagttcgaagttagctggcgtaaaatcagcaccgcgatttacggtgatgcgaagaacggcgtgtttaacaaagactatgatttcggttttggcaaagtgcgtcaggttaaagacctgcaaatgggtctgctgatgtatctgggcaagccgaaagtcgacaaacaccaccaccaccaccac

SEQ ID NO: 12 - protein, 5 aa, synthetic construct
CNGRC

SEQ ID NO: 13 - protein, 4 aa, synthetic construct
RGDS

SEQ ID NO: 14 - protein, 4 aa, synthetic construct
GRGD

SEQ ID NO: 15 - protein, 5 aa, synthetic construct
GRGDS

SEQ ID NO: 16 - protein, 6 aa, synthetic construct
GRGDSP

SEQ ID NO: 17 - protein, 7 aa, synthetic construct
GRGDSPK

SEQ ID NO: 18 - protein, 6 aa, synthetic construct
GRGDNP

SEQ ID NO: 19 - protein, 6 aa, synthetic construct
GRGDTP

SEQ ID NO: 20 - DNA, 855 bp, synthetic construct
  source 1..855: mol_type = other DNA
catatggcttgtgattgtaggggagattgtttttgtggagggggatccagtagatcaagcgtgaccagcatcaccctggacctggttaacccgaccgcgggccagtacagcagctttgtggacaagattcgtaacaacgttaaggacccgaacctgaaatatggcggtaccgatatcgcggtgattggtccgccgagcaaggagaaattcctgcgtatcaactttcaaagcagccgtggcaccgttagcctgggtctgaagcgtgacaacctgtacgtggttgcgtatctggcgatggataacaccaacgtgaaccgtgcgtactatttcaaaagcgagattaccagcgcggaactgaccgcgctgtttccggaagcgaccaccgcgaaccagaaggcgctggagtacaccgaagactatcaaagcatcgagaaaaacgcgcagattacccaaggtgacaagagccgtaaagaactgggcctgggtatcgatctgctgctgaccttcatggaggcggttaacaagaaagcgcgtgtggttaagaacgaagcgcgtttcctgctgatcgcgattcagatgaccgcggaagtggcgcgttttcgttacatccaaaacctggttaccaagaacttcccgaacaaatttgacagcgataacaaagtgattcagttcgaagttagctggcgtaaaatcagcaccgcgatttacggcgatgcgaagaacggtgtgtttaacaaagactatgatttcggctttggcaaagtgcgtcaggttaaagacctgcaaatgggcctgctgatgtatctgggcaagccgaaagtcgacaaacaccaccaccaccaccactaagaattc

SEQ ID NO: 21 - DNA, 840 bp, synthetic construct
  source 1..840: mol_type = other DNA
catatgtgtaatggaaggtgtggagggggatccagtagatcaagtgtaacatcaataaccctggacctggttaacccgaccgcgggccagtacagcagcttcgtggacaagattcgtaacaacgttaaggacccgaacctgaaatatggcggtaccgatatcgcggtgattggtccgccgagcaaggagaaattcctgcgtatcaactttcaaagcagccgtggcaccgttagcctgggtctgaagcgtgacaacctgtacgtggttgcgtatctggcgatggataacaccaacgtgaaccgtgcgtactatttcaaaagcgagattaccagcgcggaactgaccgcgctgtttccggaagcgaccaccgcgaaccagaaggcgctggagtacaccgaagactatcaaagcatcgagaaaaacgcgcagattacccaaggtgacaagagccgtaaagaactgggcctgggtatcgatctgctgctgacctttatggaggcggttaacaagaaagcgcgtgtggttaagaacgaagcgcgtttcctgctgatcgcgattcagatgaccgcggaagtggcgcgttttcgttacatccaaaacctggttaccaagaacttcccgaacaaatttgacagcgataacaaagtgattcagttcgaagttagctggcgtaaaatcagcaccgcgatttacggcgatgcgaagaacggtgtgtttaacaaagactatgatttcggctttggcaaagtgcgtcaggttaaagacctgcaaatgggcctgctgatgtatctgggcaagccgaaagtcgacaaacaccaccaccaccaccactaagaattc

SEQ ID NO: 22 - DNA, 897 bp, synthetic construct
  source 1..897: mol_type = other DNA
catatgtttgaaacactaaggggagatgaaagaatattatcaatactaaggcaccaaaacctgctgaaggagctgcaagacggtggatccagccgtagcagcgtgaccagcatcaccctggatctggttaacccgaccgcgggccagtacagcagctttgtggacaaaattcgtaacaacgttaaggacccgaacctgaaatatggtggcaccgatatcgcggtgattggtccgccgagcaaggagaaattcctgcgtatcaactttcaaagcagccgtggtaccgttagcctgggcctgaagcgtgacaacctgtacgtggttgcgtatctggcgatggataacaccaacgtgaaccgtgcgtactatttcaaaagcgagattaccagcgcggaactgaccgcgctgtttccggaagcgaccaccgcgaaccagaaggcgctggagtacaccgaagactatcaaagcatcgagaaaaacgcgcagattacccaaggcgacaagagccgtaaagaactgggtctgggcatcgatctgctgctgaccttcatggaggcggttaacaagaaagcgcgtgtggttaagaacgaagcgcgtttcctgctgatcgcgattcagatgaccgcggaagtggcgcgttttcgttacatccaaaacctggttaccaagaacttcccgaacaaatttgacagcgataacaaagtgattcagttcgaagttagctggcgtaaaatcagcaccgcgatttacggtgatgcgaagaacggcgtgtttaacaaagactatgatttcggttttggcaaagtgcgtcaggttaaagacctgcaaatgggtctgctgatgtatctgggcaagccgaaagtcgacaaacaccaccaccaccaccactaagaattc

SEQ ID NO: 23 - protein, 274 aa, synthetic construct
ACDCRGDCFCGGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKVDK

SEQ ID NO: 24 - protein, 271 aa, synthetic construct
ACDCRGDCFCGGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPK

SEQ ID NO: 25 - protein, 269 aa, synthetic construct
CNGRCGGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKVDK

SEQ ID NO: 26 - protein, 266 aa, synthetic construct
CNGRCGGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPK

SEQ ID NO: 27 - protein, 288 aa, synthetic construct
FETLRGDERILSILRHQNLLKELQDGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKVDK

SEQ ID NO: 28 - protein, 285 aa, synthetic construct
FETLRGDERILSILRHQNLLKELQDGGSSRSSVTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPK

SEQ ID NO: 29 - protein, 10 aa, synthetic construct
GGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 25 aa, synthetic construct
GGGGSGGGGSGGGGSGGGGSGGGGS

SEQ ID NO: 33 - protein, 16 aa, synthetic construct
SGGGGSGGGGSGGGGS

SEQ ID NO: 34 - protein, 15 aa, synthetic construct
GGGGSGGGGSGGGAS

SEQ ID NO: 35 - protein, 5 aa, synthetic construct
GGGGS

SEQ ID NO: 36 - protein, 7 aa, synthetic construct
SCNGRCG

SEQ ID NO: 37 - protein, 253 aa, Saponaria officinalis
VTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAELTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 38 - protein, 457 aa, Homo sapiens [sequence omitted]

SEQ ID NO: 39 - protein, 6 aa, synthetic construct
CGGSGG

SEQ ID NO: 40 - protein, 6 aa, synthetic construct
HHHHHH

(19) United States
(12) Patent Application Publication
(10) Pub. No.: US 2025/0215411 A1
(43) Pub. Date: Jul. 3, 2025

(54) FUSION PROTEINS AND USES THEREOF

(71) Applicant: OSPEDALE SAN RAFFAELE SRL, Milano (IT)

(72) Inventor: Riccardo VAGO, Milano (IT)

(73) Assignee: OSPEDALE SAN RAFFAELE SRL, Milano (IT)

(21) Appl. No.: 18/844,832

(22) PCT Filed: Mar. 9, 2023
(86) PCT No.: PCT/EP2023/056018

§ 371 Date: Sep. 6, 2024

(30) Foreign Application Priority Data
Mar. 9, 2022 (IT) 102022000004556

(51) Int. Cl. C12N 9/24; A61K 38/00; A61K 45/06; A61P 35/00; C07K 14/435

(52) U.S. Cl. CPC C12N 9/2497; A61K 45/06; A61P 35/00; C07K 14/435; A61K 38/00; C07K 2319/01; C07K 2319/21; C12Y 302/02

(57) ABSTRACT

The present invention refers to: a) at least one peptide recognizing tumor endothelial cell markers, the peptide being preferably: —an αv-integrin ligand, preferably a ligand of αvβ3, βvβ5, αvβ8, α5β1 and/or αvβ6 integrin, more preferably the peptide includes a sequence having a RGD motif or of a CgA sequence or—a ligand of CD 13 receptor, more preferably the ligand includes a sequence having a NGR motif or functional fragments or derivatives or a biologically active variant thereof and b) saporin or functional fragments, derivatives or a biologically active variant thereof.

The present invention refers to a fusion protein or conjugate comprising at least one peptide (or ligand) recognizing tumor endothelial cell markers and saporin or functional fragments, derivatives or a biologically active variant thereof, and to their medical uses.

### PRIOR ART

Toxic molecules produced by plants have been assumed to be part of their defense weapons, even if in some cases a few of them are present in edible plants, including species that are eaten raw. From an evolutionary point of view, the selective pressure by the environment on plants has led to the development and optimization of highly efficient toxin molecules, which may represent potent and efficient cytotoxic agents that can be potentially exploited in cancer therapy. Their small size, high molecular stability, easy of production, high potency and direct cell-killing property make this class of cytotoxic agents very attractive for the development of new anti-cancer therapies [1-4]. Among the various toxins so far studied for this purpose, the plant-derived type I ribosome inactivating proteins (RIPs) represent ideal candidates, owing to their high efficiency in irreversibly inhibiting protein translation and causing prompt cell death [2, 5]. Furthermore, type I RIPs consist only of an N-glycosidase domain, lacking the lectin domain, typical of type II RIP, which bind galactose residues on the cell surface and facilitates the catalytic portion to enter the cell. This feature offers the opportunity to couple type I RIPs with a tumor-targeting ligand that enable specific and selective delivery of the toxins to cancer cells, thereby improving their therapeutic index. According to this view, type I RIPs have been coupled to growth factors or other polypeptides capable of recognizing receptors over-expressed on the surface of cancer cells or on tumor endothelial cells [2, 6-8].

Saporin (SAP), a type I RIP characterized by unusual resistance to high temperature, denaturation and proteolysis and by a strong intrinsic cytotoxic activity, may represent a suitable candidate for the design and development of new anti-cancer drugs. Recent studies have shown that coupling SAP with tumor targeting ligands, such as monoclonal antibodies, peptides and aptamers, improves its cytotoxic effects on different cancer types, both vitro and in vivo [9-14]. In particular, SAP-based, chimeric recombinant proteins formed by the toxin fused to the amino-terminal fragment of urokinase [11, 13], the epidermal growth factor [12, 15], the anti-CD22 ScFv [9] have been produced and successfully tested. Thus, the development of new strategies for targeted delivery of SAP to tumors is of great experimental and pharmacological interest. At this regard, a growing body of evidence suggests that integrins may represent important molecular targets on cancer cells. In fact, the expression of certain integrins is increased on various types of cancer cells and tumor vasculature, to regulate many steps of tumor progression, such as angiogenesis and tumor cell growth, survival, migration and invasion [16-19]. For example, certain αv-integrins, such as αvβ3, αvβ6, α5β1 and αvβ5, are upregulated in various solid cancers, tumor microenvironment and upon anti-cancer therapy, while they are expressed at lower or undetectable levels in normal tissues [20]. In particular, αvβ3 and αvβ5 are known to be overexpressed in the tumor vasculature and to be involved in tumor angiogenesis [21, 22]. Integrin over-expression is associated with pathological outcomes including disease stage, metastasis formation, treatment resistance, and patient survival [20]. Thus, ligands of specific integrin subclasses may be exploited, in principle, for the development of new tumor-homing derivatives of SAP.

In the last years, many investigators have explored the potential of peptides as integrin ligands, a promising class of molecules that, owing to their small size, low immunogenicity, ease of manufacture at reasonable costs, can overcome many of the limitations related to the use of monoclonal antibodies as targeting moieties. For instance, RGD-based peptides have been widely investigated as ligands for targeted delivery of drugs and nanoparticles to tumors. In particular, ACDCRGDCFCG (RGD-4C) (SEQ ID NO: 2), a peptide capable of recognizing with high affinity αvβ3, and, although with a lower affinity, also αvβ5, α5β1 and αvβ6 (26) has proven useful to enhance the selective delivery of various types of compounds to tumors, including cytokines and toxins [23-26].

The NGR motif or sequence has been discovered in the nineties by in vivo selection of peptide-phage libraries in tumor-bearing mice (Arap, Pasqualini, and Ruoslahti 1998; Corti et al. 2008).

Systemic administration of a phage library into nude mice bearing human breast carcinoma xenografts led to the selection of tumor vasculature-homing phages carrying various peptide sequences containing this motif. Mechanistic studies showed that a cyclic disulfide-bridged peptide containing NGR (CNGRC (SEQ ID NO: 12)) can specifically recognize vessels expressing aminopeptidase N (CD13), a membrane-bound metalloproteinase that is barely or not at all expressed by normal blood vessels, but is up-regulated in angiogenic blood vessels (Curnis et al. 2002; Pasqualini et al. 2000; Lahdenranta et al. 2007; Buehler et al. 2006). This protease has a role in protein degradation, cytokine regulation, antigen presentation, cell proliferation, cell migration, and angiogenesis (Curnis et al. 2002; Mina-Osorio 2008; Luan and Xu 2007; Bhagwat et al. 2001). In tumor tissues, CD13 is expressed by endothelial cells and pericytes, and, in some cases, by tumor cells and fibroblasts. CD13 is also expressed by many cells of normal tissues, including epithelial cells from the small intestine, proximal renal tubules, prostate, bile duct canaliculi, keratinocytes, mast cells, myeloid cells, and antigen-presenting cells (Curnis et al. 2002; Taylor 1993; Shipp and Look 1993; Dixon et al. 1994; Di Matteo et al. 2010). Immunohistochemical and biodistribution studies showed that CNGRC-containing compounds bind CD13-positive tumor blood vessels, but not other CD13-rich tissues (Curnis et al. 2002; Curnis et al. 2000). Direct binding assays with a CNGRCG-TNF conjugate (NGR-TNF) and competitive binding assays with anti-CD13 antibodies showed that a CD13 form expressed by tumor blood vessels could function as a vascular receptor for the NGR motif. In contrast, CD13 expressed in normal kidney and in myeloid cells failed to bind to NGR-TNF. Consistently with these results, neither radiolabeled ¹²⁵I-NGR-TNF nor ¹²⁵I-TNF accumulated in normal organs containing CD13-expressing cells after administration to mice (Curnis et al. 2002). It would appear, therefore, that a functionally active form of CD13, in terms of NGR binding, is present in the tumor vasculature, but not in other CD13-rich tissues. The structural basis of this NGR selectivity is still unknown. The recognition of angiogenic blood vessels by NGR has been demonstrated also with cyclic-NGR-labeled paramagnetic quantum dots and quantitative molecular magnetic resonance imaging in tumor mouse models (Oostendorp et al. 2008). Ex-vivo two-photon laser scanning microscopy showed that these particles bind primarily to the endothelial lining of tumor vessels.

Bladder cancer (BC) is among the most common and expensive types of cancer in developed countries. Initial treatments rely on surgical resection of non-muscle invasive BC (NMIBC) often followed by chemotherapy, but frequently recurrences take place and many patients develop muscle invasive BC with unfavorable prognosis. Initial treatment of NMIBC relies on trans-urethral-resection (TUR) followed by an intravesical instillation of an adjuvant chemotherapy (mitomicin-C/epirubicin). High-risk patients often undergo maintenance chemo- or immunotherapy with Bacillus Calmette-Guerin, effective in reducing recurrences (Babjuk et al. Eur. Urol. 2017), but often limited by resistance and side effects. MIBC patients are treated by radical cystectomy followed by chemotherapy, nevertheless up to 50% develop metastatic disease (Witjes et al. Eur. Urol. 2017). Alternative and/or complementary strategies to state-of-the-art therapies as well as novel diagnostic tools are urgently needed for timely and specific interventions that limit the recurrence and improve survival. So far, no targeted agents have been approved for BC treatment.

Therefore, there is still the need of therapies for cancer, such as bladder cancer.

### SUMMARY OF THE INVENTION

Although toxins may have some advantages compared to chemotherapeutic drugs in cancer therapy, e.g. a potent cytotoxic activity and a reduced risk of resistance, their successful application in the treatments to solid tumors still remains to be fully demonstrated. In this study, inventors genetically modified the structure of the plant-derived single-chain ribosome inactivating protein saporin (SAP) by fusing its N-terminus to the ACDCRGDCFCG (SEQ ID NO: 2) peptide (RGD-4C), an αv-integrin ligand, and explored the anti-tumor activity of the resulting protein (herein defined RGD-SAP) in vitro and in vivo, using a model of muscle invasive bladder cancer. Inventors found that the RGD-4C targeting domain enhances the cytotoxic activity of SAP against various tumor cell lines, in a manner dependent on αv-integrin expression levels. In a subcutaneous syngeneic model of bladder cancer, RGD-SAP significantly reduced tumor growth in a dose-dependent manner. Furthermore, systemic administration of RGD-SAP in combination with mitomycin C, a chemotherapeutic drug currently used to treat patients with bladder cancer, increased the survival of mice bearing orthotopic bladder cancer with no evidence of systemic toxicity. Overall, the results suggest that RGD-SAP represents an efficient drug that could be exploited, either alone or in combination with the state-of-the-art therapies, for the treatment of bladder cancer and, potentially, of other solid tumors.

Therefore the fusion proteins of the invention specifically and efficiently attack tumor mass, significantly impairing tumor growth and reduce side effects triggered by chemotherapeutics. They represents a novelty in the field of bladder cancer in terms of therapeutic interventions. The present saporin-based drugs may be employed also in other solid cancers, due to the possibility to target primarily tumor vasculature, but also tumor cells expressing target receptors.

### DESCRIPTION OF THE INVENTION

Toxins of bacterial or plant origin have been used in recent decades as anti-tumor therapeutics with promising results in pre-clinical and clinical studies. Saporin (SAP) is a ribosome inactivating protein derived from Saponaria Officinalis, which exerts its activity by cutting a conserved region in the rRNA of the major ribosome subunit, causing irreversible inhibition of protein synthesis and, thus, cell death by apoptosis. SAP is likely to be a good therapeutic candidate because it is very stable and has a good enzymatic activity, which is preserved also when conjugated of fused to antibodies and peptides. To confer selectivity to tumor cells and so limit unspecific effects, targeting domains recognizing molecules exclusively expressed or overexpressed on tumor cells can be added to SAP. To this purpose, inventors have prepared three different constructs by fusing the SAP gene with the coding sequences of small peptides (NGR motif, RGD-4C and a chromogranin A loop) to direct the toxin towards the CD13 receptors and integrins αvβ3, αvβ5 and αvβ6, mainly present on tumor neo-vessels, but also on tumor cells. Sequences encoding the SAP-based chimeric fusions, optimized for prokaryotic expression were cloned and expressed in bacteria after induction. The conditions of production were optimized to minimize self-intoxication, while allowing sustained production. Proteins were then purified in two steps: first through affinity chromatography, by exploiting the His tail, and then through ion exchange chromatography, by taking advantage of the high isoelectric point of SAP. The cytotoxic activity of each recombinant protein was than tested in bladder cancer cell lines resembling different stages of the disease and in glioblastoma cells as prototypes for the expression of different integrins. All proteins resulted cytotoxic, demonstrating that the insertion of targeting moieties did not interfere with the SAP activity. The recombinant proteins were more toxic against cells overexpressing the integrins, whereas they were less active against healthy fibroblast, lacking them. The specificity was confirmed by using an untargeted SAP, which resulted less cytotoxic. In conclusion, inventors have developed a strategy for the production of bioactive recombinant proteins directed towards molecules expressed on tumor cells and vessels.

The present inventors therefore tested the hypothesis that fusing RGD-4C to SAP, by recombinant DNA technology, can increase its tumor selectivity and therapeutic activity. Inventors show that the RGD-SAP conjugate can be easily produced in E. coli with no need of renaturation, and that this product can kill integrin-expressing cells more efficiently than a SAP variant lacking the RGD domain. Moreover, inventors show that RGD-SAP can inhibit the tumor growth in mouse models of bladder cancer.

Inventors employed small peptides ranking from 6 (NGR) to 25 (CgA) amino acids as targeting moieties, which were fused to saporin to generate small molecular weight products (around 30 kDa), much less prone to degradation, more stable and suited to penetrate the tumor mass.

The recombinant proteins of the invention can be used as a tool for selectively killing cells based on cell surface molecules, e.g. they can be used for the treatment of solid cancers by exploiting the activity of saporin-based fusion proteins on transformed cells and tumor neo-vasculature expressing such markers. The possibility to target the tumor microenvironment, vessels in particular, renders them suitable to hit vascularized solid tumors, meaning a broad spectrum of action. Other diseases characterized by the formation of new vessels can be considered as target of the present recombinant proteins.

It is therefore an object of the present invention a fusion protein or conjugate comprising:
a) at least one peptide recognizing tumor endothelial cell markers, said peptide being preferably:
   an αv-integrin ligand, preferably a ligand of αvβ3, αvβ5, αvβ8, α5p 1 and/or αvβ6 integrin, more preferably said peptide comprising or consisting of a sequence comprising a RGD motif or of a CgA sequence
   or
   a ligand of CD13 receptor, more preferably said ligand comprising or consisting of a sequence comprising a NGR motif or functional fragments or derivatives or a biologically active variant thereof and
b) saporin or functional fragments, derivatives or a biologically active variant thereof.

The fusion protein or conjugate is preferably able to target tumor endothelial cell marker(s).

The saporin has preferably at least 80% sequence identity with, or comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK).

The saporin has preferably at least 80% sequence identity with, or comprises or consists of SEQ ID NO: 37. The saporin preferably comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK) or of SEQ ID NO: 37.

The sequence comprising a RGD motif has preferably at least 80% sequence identity with, or comprises or consists of SEQ ID NO: 2 (ACDCRGDCFCG), more preferably it comprises or consists of SEQ ID NO:2.

The CgA sequence is preferably CgA39-63. Preferably it has at least 80% sequence identity with, or comprises or consists of SEQ ID NO: 3 (FETLRGDERILSILRHQNLLKELQD), more preferably it comprises or consists of SEQ ID NO: 3.

The sequence comprising a NGR motif has preferably at least 80% sequence identity with, or comprises or consists of SEQ ID NO: 4 (CNGRCG), preferably it comprises or consists of SEQ ID NO: 4.

The fusion protein or conjugate of the invention preferably further comprises a linker component between a) and b) of claim 1, said linker preferably comprises at least 3 amino acids, it preferably has at least 90% sequence identity with SEQ ID NO: 5 (GGSSRSS), more preferably it comprises or consists of SEQ ID NO: 5 (GGSSRSS).

The fusion protein or conjugate of the invention preferably comprises from N-terminus to C-terminus the items a) and b) disclosed above.

Preferably the fusion protein or conjugate of the invention further comprises a spacer sequence at the C-terminus, more preferably said sequence is VDK. Preferably the fusion protein or conjugate of the invention further comprises an histidine tag, preferably comprising 6 histidines, at the C-terminus, preferably after the VDK or spacer sequence.

Preferably, the fusion protein or conjugate comprises from N-terminus to C-terminus:
i. a peptide recognizing tumor endothelial cell markers comprising or consisting of a sequence comprising a RGD motif having at least 80% sequence identity with, or comprising or consisting of SEQ ID NO: 2 (ACDCRGDCFCG) or functional fragments or derivatives or a biologically active variant thereof and
ii. saporin or functional fragments, derivatives or a biologically active variant thereof.

Preferably, the saporin has at least 80% sequence identity with, or comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK).

Preferably the fusion protein or conjugate is able to target tumor endothelial cell markers and:
the sequence comprising a RGD motif having at least 80% sequence identity with SEQ ID NO: 2 (ACDCRGDCFCG) or the functional fragments or derivatives or a biologically active variant thereof present the same activity of SEQ ID NO:2 and
the saporin having at least 80% sequence identity with SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK) or functional fragments, derivatives or a biologically active variant thereof present the same activity of saporin.

Preferably the sequence comprising a RGD motif comprises or consists of SEQ ID NO:2.

Preferably the sequence comprising a RGD motif comprises or consists of SEQ ID NO:2 or functional fragments, derivatives or a biologically active variant thereof and the saporin comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK) or functional fragments, derivatives or a biologically active variant thereof.

Preferably the sequence comprising a RGD motif comprises or consists of SEQ ID NO:2 and the saporin comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK).

Preferably, the fusion protein or conjugate further comprises a linker component between the peptide recognizing tumor endothelial cell markers and saporin, wherein the linker preferably comprises at least 3 amino acids.

Preferably the linker has at least 90% sequence identity with SEQ ID NO: 5 (GGSSRSS), more preferably it comprises or consists of SEQ ID NO: 5 (GGSSRSS).

The fusion protein or conjugate of the invention preferably has at least 90% sequence identity with, or comprising or consisting of

SEQ ID NO: 6
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDKHHHHHH),
SEQ ID NO: 23
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDK),
SEQ ID NO: 24
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPK),
SEQ ID NO: 7
(CNGRCGGGSSRSSVTSITLDLVNPTAGQYSSFVDKIRNNVKDPNLKYGGTDIAVIGPPSK
EKFLRINFQSSRGTVSLGLKRDNLYVVAYLAMDNTNVNRAYYFKSEITSAELTALFPEA
TTANQKALEYTEDYQSIEKNAQITQGDKSRKELGLGIDLLLTFMEAVNKKARVVKNEA
RFLLIAIQMTAEVARFRYIQNLVTKNFPNKFDSDNKVIQFEVSWRKISTAIYGDAKNGVF
NKDYDFGFGKVRQVKDLQMGLLMYLGKPKVDKHHHHHH),
SEQ ID NO: 25
(CNGRCGGGSSRSSVTSITLDLVNPTAGQYSSFVDKIRNNVKDPNLKYGGTDIAVIGPPSK
EKFLRINFQSSRGTVSLGLKRDNLYVVAYLAMDNTNVNRAYYFKSEITSAELTALFPEA
TTANQKALEYTEDYQSIEKNAQITQGDKSRKELGLGIDLLLTFMEAVNKKARVVKNEA
RFLLIAIQMTAEVARFRYIQNLVTKNFPNKFDSDNKVIQFEVSWRKISTAIYGDAKNGVF
NKDYDFGFGKVRQVKDLQMGLLMYLGKPKVDK),
SEQ ID NO: 26
(CNGRCGGGSSRSSVTSITLDLVNPTAGQYSSFVDKIRNNVKDPNLKYGGTDIAVIGPPSK
EKFLRINFQSSRGTVSLGLKRDNLYVVAYLAMDNTNVNRAYYFKSEITSAELTALFPEA
TTANQKALEYTEDYQSIEKNAQITQGDKSRKELGLGIDLLLTFMEAVNKKARVVKNEA
RFLLIAIQMTAEVARFRYIQNLVTKNFPNKFDSDNKVIQFEVSWRKISTAIYGDAKNGVF
NKDYDFGFGKVRQVKDLQMGLLMYLGKPK),
SEQ ID NO: 8
(FETLRGDERILSILRHQNLLKELQDGGSSRSSVTSITLDLVNPTAGQYSSFVDKIRNNVK
DPNLKYGGTDIAVIGPPSKEKFLRINFQSSRGTVSLGLKRDNLYVVAYLAMDNTNVNRA
YYFKSEITSAELTALFPEATTANQKALEYTEDYQSIEKNAQITQGDKSRKELGLGIDLLLT
FMEAVNKKARVVKNEARFLLIAIQMTAEVARFRYIQNLVTKNFPNKFDSDNKVIQFEVS
WRKISTAIYGDAKNGVFNKDYDFGFGKVRQVKDLQMGLLMYLGKPKVDKHHHHHH),
SEQ ID NO: 27
(FETLRGDERILSILRHQNLLKELQDGGSSRSSVTSITLDLVNPTAGQYSSFVDKIRNNVK
DPNLKYGGTDIAVIGPPSKEKFLRINFQSSRGTVSLGLKRDNLYVVAYLAMDNTNVNRA
YYFKSEITSAELTALFPEATTANQKALEYTEDYQSIEKNAQITQGDKSRKELGLGIDLLLT
FMEAVNKKARVVKNEARFLLIAIQMTAEVARFRYIQNLVTKNFPNKFDSDNKVIQFEVS
WRKISTAIYGDAKNGVFNKDYDFGFGKVRQVKDLQMGLLMYLGKPKVDK)
or
SEQ ID NO: 28
(FETLRGDERILSILRHQNLLKELQDGGSSRSSVTSITLDLVNPTAGQYSSFVDKIRNNVK
DPNLKYGGTDIAVIGPPSKEKFLRINFQSSRGTVSLGLKRDNLYVVAYLAMDNTNVNRA
YYFKSEITSAELTALFPEATTANQKALEYTEDYQSIEKNAQITQGDKSRKELGLGIDLLLT
FMEAVNKKARVVKNEARFLLIAIQMTAEVARFRYIQNLVTKNFPNKFDSDNKVIQFEVS
WRKISTAIYGDAKNGVFNKDYDFGFGKVRQVKDLQMGLLMYLGKPK),

or functional fragments, equivalents, variants, mutants, derivatives, synthetics or recombinants functional analogues thereof.

Preferably the fusion protein or conjugate comprises or consists of:

SEQ ID NO: 6
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDKHHHHHH)
or
SEQ ID NO: 23
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDK)
or
SEQ ID NO: 24
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPK)

or functional fragments, equivalents, variants, mutants, derivatives, synthetics or recombinants functional analogues thereof.

Preferably the fusion protein or conjugate of any one of previous claims comprises or consists of:

SEQ ID NO: 6
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDKHHHHHH)
or
SEQ ID NO: 23
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDK)
or
SEQ ID NO: 24
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPK).

A further object of the invention is an isolated nucleic acid encoding the fusion protein or conjugate or functional fragments, equivalents, variants, mutants, derivatives, synthetics or recombinants functional analogues thereof as disclosed above.

A further object is a recombinant expression vector comprising the isolated nucleic above disclosed operably linked to regulatory sequences capable of directing expression of a gene encoding said fusion protein in a host.

Another object of the invention is a host cell comprising and/or expressing the fusion protein or conjugate or functional fragments, equivalents, variants, mutants, derivatives, synthetics or recombinants functional analogues thereof of the present invention, or the isolated nucleic acid or the recombinant expression vector of the present invention, said host cell preferably being selected from the group consisting of:
fungal cells, bacterial cells, preferably gram-positive Bacilli such as B. subtilis, B. licheniformis, B. megaterium, B. amyloliquefaciens, B. pumilus, gram negative bacteria such as Escherichia coli, plant protoplasts, unicellular algae, actinomycetales such as Streptomyces sp., and yeasts, such as Saccharomyces cerevisiae, Pichia pastoris, Yarrowia lipolytica, most preferably Trichoderma reesei or Bacillus.

A further object of the invention is a method of producing the fusion protein or conjugate or functional fragments, equivalents, variants, mutants, derivatives, synthetics or recombinants functional analogues thereof of the invention comprising the steps of transforming a host cell with an expression vector encoding said fusion protein, culturing said host cell under conditions enabling expression of said fusion protein, and optionally recovering and purifying said fusion protein.

Another object of the invention is a composition comprising the fusion protein or conjugate or functional fragments, equivalents, variants, mutants, derivatives, synthetics or recombinants functional analogues thereof as defined herein, or a pharmaceutical composition comprising the fusion protein or conjugate or functional fragments, equivalents, variants, mutants, derivatives, synthetics or recombinants functional analogues thereof as defined herein and at least one pharmaceutically acceptable carrier, preferably further comprising an anti-cancer agent, e.g. a chemotherapeutic drug, such as a mitomycin C.

A further object if the invention is a fusion protein or conjugate or functional fragments, equivalents, variants, mutants, derivatives, synthetics or recombinants functional analogues thereof as defined herein for medical use, preferably for use in the treatment of cancer, preferably vascularized solid tumors or irrorated solid tumors, preferably bladder cancer, lung cancer, breast cancer, colorectal cancer, ovarian cancer prostate cancer or pancreatic cancer, or for use in the treatment of pathologies in which there is a pathological proliferation of blood vessels.

Further objects of the invention are a nucleic acid encoding for the fusion protein or conjugate or functional fragments, equivalents, variants, mutants, derivatives, synthetics or recombinants functional analogues thereof as above defined, a vector, preferably a plasmid or a viral vector, preferably for gene therapy containing the said nucleic acid, and a nanoparticle comprising the fusion protein or conjugate as above defined, preferably the protein is adsorbed on the surface of gold nanoparticles.

Another object of the invention is a combination product comprising the fusion protein or conjugate or functional fragments, equivalents, variants, mutants, derivatives, synthetics or recombinants functional analogues thereof as above defined or the nucleic acid as above defined or the vector as above defined or the nanoparticle as above defined and at least one antitumor agent, preferably being a chemotherapeutic agent and/or immunomodulator and/or autoimmune cell. Preferably the chemotherapeutic agent is mitomycin C, doxorubicin, melphalan, temozolomide, gemcitabine, taxol, cisplatin, vincristine, or vinorelbine. Preferably the further antitumor agent comprises an antibody and a chemotherapeutic agent, such as R-CHOP: rituximab, cyclophosphamide, vincristine, doxorubicin, prednisolone.

The fusion proteins or conjugates or functional fragments, equivalents, variants, mutants, derivatives, synthetics or recombinants functional analogues thereof or the combination product or the nucleic acid or the vector or the nanoparticle as above defined are preferably for medical use, more preferably for use in the treatment of tumors, preferably solid tumors, preferably vascularized solid tumors or irrorated solid tumors, preferably bladder cancer, lung cancer, breast cancer, colorectal cancer, ovarian cancer, prostate cancer or pancreatic cancer, lymphomas, preferably primary diffuse large B-cell lymphoma of the CNS (PCNSL), brain tumors (e.g. glioma, astrocytoma, glioblastoma, diffuse intrinsic pontine glioma), sarcoma, melanoma oral or skin squamous cell carcinoma, hepatocellular carcinoma, head and neck, gastroesophageal, lung (e.g. SCLC, NSCLC, mesothelioma), cervix, renal, urothelial or metastasis thereof, or for use in the treatment of pathologies in which there is a pathological proliferation of blood vessels.

Preferably at least one antitumor agent as defined above is also administered.

Another object of the invention is a pharmaceutical composition comprising an effective amount of a fusion protein or conjugates or functional fragments, equivalents, variants, mutants, derivatives, synthetics or recombinants functional analogues thereof or the nucleic acid or the vector or the combination product or the nanoparticle as above defined, and at least one pharmaceutically acceptable carrier and/or excipient. Optionally the pharmaceutical composition further comprises at least one antitumor agent.

A further object of the invention is a method for producing the fusion protein or conjugates or functional fragments, equivalents, variants, mutants, derivatives, synthetics or recombinants functional analogues thereof of the invention, said method comprising: the expression of a conjugate as defined above in prokaryotic or eukaryotic cells, preferably E. coli cells.

In the context of the present invention the fusion protein also includes functional fragments, equivalents, variants, mutants, derivatives, synthetics or recombinants functional analogues thereof or conjugates or recombinant proteins or functional fragments, equivalents, variants, mutants, derivatives, synthetics or recombinants functional analogues thereof. In another embodiment the conjugate or fusion protein is in the form of nucleic acid, a plasmid or a viral vector for gene therapy. In another embodiment the fusion protein or conjugate is in the form of a nanoparticle, e.g. adsorbed on the surface of gold nanoparticles

In a preferred embodiment the composition further comprises another antitumor agent.

Preferably the further antitumor agent is a chemotherapeutic drug, or an immunomodulator, or immune cells. Preferably the chemotherapeutic drug is mitomycin C, doxorubicin, melphalan, gemcitabine, taxol, cisplatin, vincristine, or vinorelbine.

Other objects of the invention are an expression vector comprising the nucleic acid as defined above, a host cell transformed with said expression vector and a method for preparing a conjugate or fusion protein as defined above comprising culturing said host cell under conditions which provide for the expression of the conjugate or of the fusion protein.

In the context of the present invention saporin (or SAP) is intended or may be defined as a ribosome inactivating protein.

In the context of the present invention the mentioned peptides, polypeptides or proteins are preferably in a mature form.

The term “ligand” includes an agent able to recognize and/or bind a target, e.g. tumor endothelial cell markers, preferably CD13 receptor (or CD13).

The term “CD13 receptor” includes also “CD13”.

The peptides recognizing tumor endothelial cell markers of the invention may be a ligand.

The linker of the invention must allow the targeting domain to be recognized by its receptor because it is not too close to saporin, risking steric hindrance problems. The linker of the invention is preferably flexible and inert. The GGSSRSS (SEQ ID NO: 5) sequence used in the present invention derives from the linker between the VH and V, subunits of the antibodies.

The terms “linker” and “spacer” are used indistinctively in the present application. A linker provides spatial separation between the different domain of the fusion proteins or conjugates of the invention. The linker may be a flexible linker. Non-limiting examples of flexible linkers that may be used in the invention include:

(SEQ ID NO: 29: GGGGSGGGGS)
-(Gly4Ser)2;
(SEQ ID NO: 30: GGGGSGGGGGGGGS)
-(Gly4Ser)3;
(SEQ ID NO: 31: GGGGS GGGGS GGGGS GGGGS)
-(Gly4Ser)4;
(SEQ ID NO: 32: GGGGGGGGSGGGGSGGGGSGGGGS)
-(Gly4Ser)5;
(SEQ ID NO: 33)
-S GGGGS GGGGS GGGGS;
(SEQ ID NO: 34)
-GGGGSGGGGSGGGAS.

The linker is preferably between 2 or 3 and 10 amino acids in length. A glycine-serine doublet provides a particularly suitable linker.

The cells of the invention are preferably E. coli or Pichia pastoris, protoplasts of tobacco (see e.g. Marshall et al. The Plant Journal 2011) and unicellular algae (see e.g. Zuppone et al. Biomedicines 2019).

The present fusion proteins may be used in combination with other toxins and other recombinant proteins with a different mechanism of action (e.g. RGD-SAP may be combined with ATF-SAP or NGR-TNF, e.g. a CNGRCG-TNF or a SCNGRCG (SEQ ID NO:36)-TNF conjugate, in particular as disclosed in WO2021186071, herein incorporated by reference), small molecules, chemotherapies (methotrexate, tetracyclines, 5-Fluorouracil, platinum compounds, taxol, etc.), immunotherapy (anti-PDL1, BCG), monoclonal antibodies . . . .

The present invention relates to a conjugate or fusion protein which is a molecule comprising at least one targeting moiety/polypeptide linked to saporin formed through genetic fusion or chemical coupling. By “linked” inventors mean that the first and second sequences are associated such that the first sequence (saporin) is able to be transported by the second sequence to a target cell. Thus, conjugates include fusion proteins in which the transport protein is linked to saporin via their polypeptide backbones through genetic expression of a DNA molecule encoding these proteins, directly synthesized proteins and coupled proteins in which pre-formed sequences are associated by a cross-linking agent. The term is also used herein to include associations, such as aggregates, of saporin with the targeting peptide/protein. The conjugates of the present invention are capable of being directed to a cell so that an effector function corresponding to the polypeptide sequence coupled to the transport sequence can take place. The peptide (such as the NGR or RGD or CgA) can be coupled directly to the saporin or preferably indirectly through a spacer or linker, which can be a single amino acid, an amino acid sequence or an organic residue, such as 6-aminocapryl-N-hydroxysuccinimide.

The peptide is preferably linked to amino acid residues which are amido- or carboxylic-bond acceptors, which may be naturally occurring on the molecule or artificially inserted using genetic engineering techniques. The modified saporin is preferably prepared by use of a cDNA comprising a 5′-contiguous sequence encoding the peptide. According to a preferred embodiment, there is provided a conjugation product between saporin and the NGR or RGD or CgA sequence in which the amino-terminal of saporin is linked to the NGR or RGD or CgA peptide through the spacer G (glycine).

The cDNA coding for the conjugate of the present invention, or for the protein may be codon optimized for the expression in the host.

Preferably, the conjugate or fusion protein according to the invention comprises from the N-terminus to the C-terminus: the NGR or RGD or CgA peptide or sequence and saporin.

The above NGR or RGD or CgA peptide or sequence may be directly linked to the N-terminus of a protein (e.g. saporin) or linked through a linker.

Preferably the at least one peptide recognizing tumor endothelial cell markers is defined as a sequence comprising a RGD motif or a CgA sequence or a sequence comprising a NGR motif or an αv-integrin ligand or a ligand of CD13 receptor.

Preferably the saporin comprises or consists of the SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK).

Saporin is preferably mature saporin.

The saporin may be any sequence known to the skilled man and may also be modified in its C-terminus e.g. by introducing a KDEL signal.

The term NGR peptide or sequence is interchangeable with the following terms “sequence comprising a NGR motif” or “CNGRC” or “NGR motif”.

The term Chromogranin A (CgA) peptide or sequence is interchangeable with the following terms “CgA sequence” or “CgA39-63”.

The term RGD peptide or sequence is interchangeable with the following terms “sequence comprising a RGD motif” or “RGD-4C” or “RGD motif”. The RGD peptide also includes the CgA peptides herein disclosed.

Preferably the RGD motif is a sequence comprising RGD.

Preferably the RGD peptide or sequence comprises or consists of SEQ ID NO: 2 (ACDCRGDCFCG). In the present invention, the RGD peptide or the integrin ligand may be any RGD peptide or integrin ligand known to the skilled man. It may also be selected from any of the ligand shown in Kapp et al., “A Comprehensive Evaluation of the Activity and Selectivity Profile of Ligands for RGD-binding Integrins”, Sci Rep 2017, herein incorporated by reference, in particular in Tables 1 and 2. E.g. it may be comprise or consists of the following sequences: RGD, RGDS (SEQ ID NO:13), GRGD (SEQ ID NO:14), GRGDS (SEQ ID NO:15), GRGDSP (SEQ ID NO:16), GRGDSPK (SEQ ID NO:17), GRGDNP (SEQ ID NO:18), GRGDTP (SEQ ID NO:19), RGD-4C, . . . .

Preferably the CgA peptide or sequence comprises or consists of SEQ ID NO: 3 (FETLRGDERILSILRHQNLLKELQD). In the present invention, the CgA peptide may be any peptide known to the skilled man. It may also be selected from any of the peptide shown in Curnis et al., “Chromogranin A binds to αvβ6-integrin and promotes wound healing in mice”, Cell. Mol. Life Sci. 2012, herein incorporated by reference.

Preferably the NGR motif is a sequence comprising NGR.

Preferably the NGR peptide or sequence comprises or consists of SEQ ID NO: 4 (CNGRCG). In the present invention, the NGR peptide may be any peptide known to the skilled man, also cyclic peptides, e.g. peptides derived from isoDGR as the ones disclosed in Curnis et al., “IsoDGR-Tagged Albumin: A New αvβ3 Selective Carrier for Nanodrug Delivery to Tumors”, Small 2013 herein incorporated by reference.

Preferably, the peptide recognizing tumor endothelial cell markers may be defined as a peptide comprising or consisting of:
a sequence having at least 80% sequence identity with, or comprising or consisting of SEQ ID NO: 2 (ACDCRGDCFCG), or functional fragments or derivatives or a biologically active variant thereof, preferably the sequence comprises or consists of SEQ ID NO: 2; or
a sequence having at least 80% sequence identity with, or comprising or consisting of SEQ ID NO: 3 (FETLRGDERILSILRHQNLLKELQD), or functional fragments or derivatives or a biologically active variant thereof, preferably the sequence comprises or consists of SEQ ID NO: 3; or
a sequence having at least 80% sequence identity with, or comprising or consisting of SEQ ID NO: 4 (CNGRCG), or functional fragments or derivatives or a biologically active variant thereof, preferably the sequence comprises or consists of SEQ ID NO: 4;
a sequence of any of the ligand shown in Kapp et al., “A Comprehensive Evaluation of the Activity and Selectivity Profile of Ligands for RGD-binding Integrins”, Sci Rep 2017, herein incorporated by reference, in particular in Tables 1 and 2, preferably the sequence is RGD, RGDS (SEQ ID NO:13), GRGD (SEQ ID NO:14), GRGDS (SEQ ID NO:15), GRGDSP (SEQ ID NO:16), GRGDSPK (SEQ ID NO:17), GRGDNP (SEQ ID NO:18), GRGDTP (SEQ ID NO:19), RGD-4C, . . .
a sequence of any peptide shown in Curnis et al., “Chromogranin A binds to αvβ6-integrin and promotes wound healing in mice”, Cell. Mol. Life Sci. 2012, herein incorporated by reference;
a sequence of a cyclic peptides, e.g. peptides derived from isoDGR as the ones disclosed in Curnis et al., “IsoDGR-Tagged Albumin: A New αvβ3 Selective Carrier for Nanodrug Delivery to Tumors”, Small 2013 herein incorporated by reference, or a cyclic disulfide-bridged peptide.

Instead of the VDK sequence which may be present at the C-terminus in the fusion protein of the invention other sequences may be used, e.g. the sequence GGSSRSS, GGGGS (SEQ ID NO: 35) repeated up to 3 times (Della Cristina et al. Microbial Cell Factories 2015), or any quite inert and flexible spacers. Such sequences should be able to increase the distance between saporin and the histidine tail and/or to facilitate the recognition of the latter in case of affinity purification.

Preferably, the nucleic acid encoding for the fusion protein or conjugate of the invention is comprised within, comprises or consists of one of SEQ ID NO: 9-11 or 20-22 or functional fragments, derivatives or biologically active variants thereof.

Preferably it comprises at least one of the sequence nt. 1-33 of SEQ ID NO:9, nt. 1-18 of SEQ ID NO: 10, nt. 1-75 of SEQ ID NO:11.

Preferably it comprises a linker identified by the underlined sequence in SEQ ID NO:9-11.

Preferably it comprises the sequence identified in bold in SEQ ID NO:9-11.

Preferably it comprises a spacer identified by the cursive sequence in SEQ ID NO:9-11.

Preferably it comprises a his tag identified by the grey sequence in SEQ ID NO:9-11.

In the method for producing a conjugate or fusion protein according to the invention, the expression of the conjugate may be carried out in any expression system known to the expert in the art.

Examples of expression systems are prokaryotic systems, such as Bacillus subtilis, E. coli, Bacillus megaterium, Lactoccos Lactis. Examples of eukaryotic expression systems are yeast systems, as e.g. Saccharomyces cerevisiae and P. pastoris, fungus systems, as e.g. Aspergillus niger and oryzae, insects systems, mammalian systems, as e.g. HEK293 and CHO, transgenic plants or animals. All the expression systems mentioned in the publication Gomes et al., Advances in Animal and Veterinary Sciences, June 2016, 4(7):346-356 are herein incorporated by reference.

A polynucleotide or nucleic acid described herein can be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual, Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. Examples of viral vectors include, for instance, adenoviral vectors, adeno-associated viral vectors, lentiviral vectors, retroviral vectors, and herpes virus vectors. A vector may be replication-proficient or replication-deficient. A vector may result in integration into a cell's genomic DNA. Typically, a vector is capable of replication in a host cell, for instance a mammalian and/or a bacterial cell, such as E. coli.

Selection of a vector depends upon a variety of desired characteristics in the resulting construct, such as a selection marker, vector replication rate, use in gene transfer into cells of the gastrointestinal tract, and the like. Suitable host cells for cloning or expressing the vectors herein are prokaryotic or eukaryotic cells. Suitable eukaryotic cells include mammalian cells, such as murine cells and human cells. Suitable prokaryotic cells include eubacteria, such as gram-negative organisms, for example, E. coli.

An expression vector optionally includes regulatory sequences operably linked to the polynucleotide of the present invention. An example of a regulatory sequence is a promoter. A promoter may be functional in a host cell used, for instance, in the construction and/or characterization of CgA polynucleotide or a fragment thereof, and/or may be functional in the ultimate recipient of the vector. A promoter may be inducible, repressible, or constitutive, and examples of each type are known in the art. A polynucleotide of the present invention may also include a transcription terminator. Suitable transcription terminators are known in the art.

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are known. Methods for in vitro synthesis also include, for instance, in vitro transcription using a circular or linear expression vector in a cell free system. Expression vectors can also be used to produce a polynucleotide of the present invention in a cell, and the polynucleotide may then be isolated from the cell.

Also provided are compositions including one or more polypeptides or polynucleotides described herein. Such compositions typically include a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration. Additional compounds can also be incorporated into the compositions.

A composition may be prepared by methods known in the art of pharmacy. In general, a composition can be formulated to be compatible with its intended route of administration. A formulation may be solid or liquid. Administration may be systemic or local. In some aspects local administration may have advantages for site-specific, targeted disease management. Local therapies may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral or rectal), and topical (e.g., epicutaneous, inhalational, transmucosal) administration. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization. Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in, for instance, ampoules, disposable syringes or multiple dose vials made of glass or plastic.

Compositions can include sterile aqueous solutions or dispersions and sterile powders for the extemporaneous preparation of sterile solutions or dispersions. For intravenous administration, suitable carriers include human albumin, physiological saline, bacteriostatic water, Cremophor EL™ (BASF, Parsippany, N.J.) or phosphate buffered saline. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, albumin, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. Sterile solutions can be prepared by incorporating the active compound (e.g., a polypeptide or polynucleotide described herein) in the required amount in an appropriate solvent with one or a combination of ingredients such as those enumerated above, as required, followed by filtered sterilization. Generally, dispersions are prepared by incorporating the active compound into a sterile vehicle, which contains a dispersion medium and other ingredient such as from those enumerated above. In the case of sterile powders for the preparation of sterile injectable solutions, methods of preparation that may be used include vacuum drying and freeze-drying which yields a powder of the active ingredient plus any additional desired ingredient from a previously sterile-filtered solution thereof.

For enteral administration, a composition may be delivered by, for instance, nasogastric tube, enema, colonoscopy, or orally. Oral compositions may include an inert diluent or an edible carrier. For the purpose of oral therapeutic administration, the active compound can be incorporated with excipients and used in the form of tablets, troches, or capsules. Oral compositions can also be prepared using a fluid carrier. Pharmaceutically compatible binding agents can be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds may be delivered in the form of an aerosol spray, a nebulizer, or an inhaler, such as a nasal spray, metered dose inhaler, or dry-powder inhaler. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds may be formulated into ointments, salves, gels, or creams as generally known in the art. An example of transdermal administration includes iontophoretic delivery to the dermis or to other relevant tissues. The active compounds can also be prepared in the form of suppositories (e.g., with conventional suppository bases such as cocoa butter and other glycerides) or retention enemas for rectal delivery. The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially. Liposomal suspensions can also be used as pharmaceutically acceptable carriers. These can be prepared according to methods known to those skilled in the art. Delivery reagents such as lipids, cationic lipids, phospholipids, liposomes, and microencapsulation may also be used.

Another object of the invention is a method of treatment and/or prevention of tumors, preferably solid tumors, more preferably lymphomas, preferably primary diffuse large B-cell lymphoma of the CNS (PCNSL), brain tumors, e.g. glioma, astrocytoma, glioblastoma, diffuse intrinsic pontine glioma, sarcoma, melanoma oral or skin squamous cell carcinoma, hepatocellular carcinoma, head and neck, gastroesophageal, colorectal, pancreatic, ovarian, lung, e.g. SCLC, NSCLC, mesothelioma, cervix, breast cancer, renal, urothelial or metastasis thereof, comprising administering the conjugate or the fusion protein or the nucleic acid or the vector or the nanoparticle or the combination product as disclosed herein to a patient in need thereof.

In the context of the present invention the term “comprising” includes the terms “comprising”, “consisting of” and “consisting essentially of”.

Included in the present invention are also nucleic acid sequences and amino acid sequences derived from the sequences shown herein and below, e.g. functional fragments, mutants, derivatives, analogues, precursors, variants and sequences having a % of identity of at least 70% with the sequences disclosed herein, and homologues of the peptides, proteins, sequences described herein. Preferably the fragments, mutants, derivatives, analogues, precursors, variants and sequences having a % of identity of at least 70% with the sequences disclosed herein, and homologues of the peptides, proteins, sequences described herein are functional and/or biologically active.

The proteins mentioned below are preferably characterized by the sequences disclosed by the corresponding NCBI accession numbers, as shown in the table and in the paragraphs below.

| Protein | NCBI Accession number |
|---|---|
| saporin | CAA48887.1 or CAA48889.1 |
| CgA₃₉₋₆₃ | AAA52018.1 |

saporin, partial [Saponaria officinalis], GenBank: CAA48887.1

| ORIGIN | |
|---|---|
| (SEQ ID NO: 1) | |
| 1 | vtsitldlvn ptagqyssfv dkirnnvkdp nlkyggtdia vigppskekf lrinfqssrg |
| 61 | tvslglkrdn lyvvaylamd ntnvnrayyf kseitsaelt alfpeattan qkaleytedy |
| 121 | qsieknaqit qgdksrkelg lgidllltfm eavnkkarvv knearfllia iqmtaevarf |
| 181 | ryiqnlvtkn fpnkfdsdnk viqfevswrk istaiygdak ngvfnkdydf gfgkvrqvkd |
| 241 | lqmgllmylg kpk |

saporin, partial [Saponaria officinalis], GenBank: CAA48889.1

| ORIGIN | |
|---|---|
| (SEQ ID NO: 37) | |
| 1 | vtsitldlvn ptagqyssfv dkirnnvkdp nlkyggtdia vigppskekf lrinfqssrg |
| 61 | tvslglkrdn lyvvaylamd ntnvnrayyf rseitsaelt alfpeattan qkaleytedy |
| 121 | qsieknaqit qgdqsrkelg lgidllstsm eavnkkarvv kdearfllia iqmtaeaarf |
| 181 | ryiqnlvikn fpnkfnsenk viqfevnwkk istaiygdak ngvfnkdydf gfgkvrqvkd |
| 241 | lqmgllmylg kpk |

//

chromogranin A [Homo sapiens], GenBank: AAA52018.1

| ORIGIN | |
|---|---|
| (SEQ ID NO: 38) | |
| 1 | mrsaavlall lcagqvtalp vnspmnkgdt evmkcivevi ydtlskpspm pvskecfetl |
| 61 | rgderilsil rhqnllkelq dlalqgrker ahqqkkhsgf edelsevlen qssqaelkqa |
| 121 | veepsskdvm ekredskeae ksgeatdgar pqalpepmqe skaegnkqap geeeeeeeea |
| 181 | tnthppaslp sqkypgpqak gdseglsqgl vdrekglsve pgwqakreee eeeeeeaeag |
| 241 | eeavpeeegp tvvlnphpsl gykeirkges rsealavdga gkpgaeeaqd pegkgeqehs |
| 301 | qqkeeeeema vvpqglfrgg ksgeleqeee rlskewedtn rwskmdqlak eltaekrleg |
| 361 | qeeeednrdr smrlsfrarg ygfrgpgpql rrgsrpnswe dsleaglplq vrgypqekke |
| 421 | eegsanrrpe deleslsai eaelekvahq lqalrrg |

//

The sequences CNGRC e RGD-4C derive from phage peptide libraries (Arap et al. Science 1998). When describing the present invention, all terms not defined herein have their common art-recognized meanings. Any term or expression not expressly defined herein shall have its commonly accepted definition understood by those skilled in the art. To the extern that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

The present invention also includes functional fragments, variants or derivatives of the proteins, peptides, conjugates or sequences herein disclosed. In the context of the present invention, when referring to specific DNA sequences, it is intended that it is comprised within the invention also RNA molecules identical to said polynucleotides, except for the fact that the RNA sequence contains uracil instead of thymine and the backbone of the RNA molecule contains ribose instead of deoxyribose, RNA sequence complementary the sequences therein disclosed, functional fragments, mutants and derivatives thereof, proteins encoded therefrom, functional fragments, mutants and derivatives thereof. The term “complementary” sequence refers to a polynucleotide which is non-identical to the sequence but either has a complementary base sequence to the first sequence or encodes the same amino acid sequence as the first sequence. A complementary sequence may include DNA and RNA polynucleotides. The term “functional” may be understood as capable of maintaining the same activity of the reference peptide, protein, sequence, . . . “Fragments” are preferably long at least 10 aa., 20 aa., 30 aa., 40 aa., 50 aa., 60 aa., 70 aa., 80 aa., 90 aa., 100 aa., . . . “Derivatives” may be recombinant or synthetic. The term “derivative” as used herein in relation to a protein means a chemically modified protein or an analogue thereof, wherein at least one substituent is not present in the unmodified protein or an analogue thereof, i.e. a protein which has been covalently modified. Typical modifications are amides, carbohydrates, alkyl groups, acyl groups, esters and the like. As used herein, the term “derivatives” also refers to longer or shorter polynucleotides/proteins and/or having e.g. a percentage of identity of at least 70%, 75%, 80%, 85%, 86%, 87%, 88%, 89%, 90%, 91%, 92%, 93%, 94%, 95%, 96%, 97%, 98%, more preferably of at least 99% with the sequences herein disclosed. In the present invention “at least 70% identity” means that the identity may be at least 70%, or 75%, or 80%, or 85% or 90% or 95% or 100% sequence identity to referred sequences. This applies to all the mentioned % of identity. In the present invention “at least 80% (sequence) identity” means that the identity may be at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to referred sequences. This applies to all the mentioned % of identity. Preferably, the % of identity relates to the full length of the referred sequence. The derivative of the invention also includes “functional mutants” of the polypeptides, which are polypeptides that may be generated by mutating one or more amino acids in their sequences and that maintain their activity. Indeed, the polypeptide of the invention, if required, can be modified in vitro and/or in vivo, for example by glycosylation, myristoylation, amidation, carboxylation or phosphorylation, and may be obtained, for example, by synthetic or recombinant techniques known in the art. Also within the scope of the subject invention are polynucleotides which have the same nucleotide sequences of a polynucleotide exemplified herein except for nucleotide substitutions, additions, or deletions within the sequence of the polynucleotide, as long as these variant polynucleotides retain substantially the same relevant functional activity as the polynucleotides specifically exemplified herein (e.g., they encode a protein having the same amino acid sequence or the same functional activity as encoded by the exemplified polynucleotide). Thus, the polynucleotides disclosed herein should be understood to include mutants, derivatives, variants and fragments, as discussed above, of the specifically exemplified sequences. The subject invention also contemplates those polynucleotide molecules having sequences which are sufficiently homologous with the polynucleotide sequences of the invention so as to permit hybridization with that sequence under standard stringent conditions and standard methods (Maniatis, T. et al, 1982). Polynucleotides described herein can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or greater as compared to a sequence exemplified herein. Unless otherwise specified, as used herein percent sequence identity and/or similarity of two sequences can be determined e.g. using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The peptide or the protein as above defined may include an amino acid sequence with at least 65%, 70%, 75%, 80%, 82%, 85%, 90%, 92%, 95%, 98%, 99% or 100% identity to the sequences herein mentioned. Determining percent identity of two amino acid sequences may include aligning and comparing the amino acid residues at corresponding positions in the two sequences. If all positions in two sequences are occupied by identical amino acid residues then the sequences are said to be 100% identical. Percent identity may be measured by the Smith Waterman algorithm (Smith T F, Waterman M S 1981 “Identification of Common Molecular Subsequences,” J Mol Biol 147: 195-197, which is incorporated herein by reference as if fully set forth). The peptide and the protein herein disclosed may have fewer or more than the residues of the mentioned sequences or may present amino acid replacement in comparison to the herein mentioned sequences. The replacement may be with any amino acid whether naturally occurring or synthetic. The replacement may be with an amino acid analogue or amino acid mimetic that functions similarly to the naturally occurring amino acids. Naturally occurring amino acids are those encoded by the genetic code, as well as those amino acids that are later modified. The later modification may be but is not limited to hydroxyproline, γ-carboxyglutamate, and O-phosphoserine modifications. Naturally occurring amino acids include the standard 20, and unusual amino acids. Unusual amino acids include selenocysteine. The replacement may be with an amino acid analogue, which refers to compounds that have the same basic chemical structure as a naturally occurring amino acid; e.g., a carbon that is bound to a hydrogen, a carboxyl group, an amino group, and an R group. Examples of amino acid analogues include but are not limited to homoserine, norleucine, methionine sulfoxide, methionine methyl sulfonium. Such analogues may have modified R groups or modified peptide backbones. The amino acid analogues may retain the same basic chemical structure as a naturally occurring amino acid. The replacement may be with an amino acid mimetic, which refers to chemical compounds that have a structure that is different from the general chemical structure of an amino acid, but that functions similarly to a naturally occurring amino acid. The replacement may be with an α, α-disubstituted 5-carbon olefinic unnatural amino acid. A replacement may be a conservative replacement, or a non-conservative replacement. A conservative replacement refers to a substitution of an amino acid with a chemically similar amino acid. Conservative substitution tables providing functionally similar amino acids are well known in the art. Such conservatively replacements include but are not limited to substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)). A replacement may be from one amino acid to another with a similar hydrophobicity, hydrophilicity, solubility, polarity, or acidity. A sequence having less than 100% identity to the mentioned sequences may be referred to as a variant. In an embodiment, one or more amino acids residues are replaced with a residue having a crosslinking moiety. As used herein, a “peptide” or “polypeptide” comprises a polymer of amino acid residues linked together by peptide (amide) bonds. The term(s), as used herein, refer to proteins, polypeptides, and peptide of any size, structure, or function. Typically, a peptide or polypeptide will be at least three amino acids long. A peptide or polypeptide may refer to an individual protein or a collection of proteins. The peptides of the instant invention may contain natural amino acids and/or non-natural amino acids (i.e., compounds that do not occur in nature but that can be incorporated into a polypeptide chain). Amino acid analogues as are known in the art may alternatively be employed. One or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof. A large number of agents are developed to target cellular contents, cellular compartments, or specific protein, lipid, nucleic acid or other targets or biomarkers within cells. While these agents can bind to their intracellular targets with strong affinity, many of these compounds, whether they be molecules, proteins, nucleic acids, peptides, nanoparticles, or other intended therapeutic agents or diagnostic markers cannot cross the cell membrane efficiently or at all.

In the context of the present invention, the sequence having a certain % of identity with the reference sequences herein identified (e.g. having at least 80% sequence identity), or the functional fragments or derivatives or a biologically active variant of the reference sequences herein identified preferably present the same activity of the reference sequence.

In particular, a functional fragment or derivative or biologically active variant of an αv-integrin ligand, preferably a ligand of αvβ3, αvβ5, αvβ8, α531 and/or αvβ6 integrin, more preferably said peptide comprising or consisting of a sequence comprising a RGD motif or of a CgA sequence thereof, is preferably able to recognize tumor endothelial cell markers and/or is an αv-integrin ligand, preferably a ligand of αvβ3, αvβ5, αvβ8, α5β1 and/or αvβ6 integrin.

A functional fragment or derivative or biologically active variant of ligand of CD13 receptor, more preferably said ligand comprising or consisting of a sequence comprising a NGR motif is preferably able to recognize tumor endothelial cell markers and/or is a ligand of CD13 receptor.

A sequence having at least 80% sequence identity with SEQ ID NO: 2 (ACDCRGDCFCG), SEQ ID NO: 3 (FETLRGDERTLSTLRHQNLLKELQD) or SEQ ID NO: 4 (CNGRCG), or the functional fragments or derivatives or a biologically active variant thereof is preferably able to recognize tumor endothelial cell markers and/or is an αv-integrin ligand, preferably a ligand of αvβ3, αvβ5, αvβ8, α5β1 and/or αvβ6 integrin and/or a ligand of CD13 receptor.

A sequence having least 80% sequence identity with saporin sequence, or a functional fragments or derivatives or a biologically active variant of saporin preferably present a cytotoxic effect against cancer cells.

The composition of the invention may include a pharmaceutically acceptable carrier. The pharmaceutically acceptable carrier may include but is not limited to at least one of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, dextrose, talc, magnesium carbonate, kaolin; non-ionic surfactants, edible oils, physiological saline, bacteriostatic water, Cremophor EL™ (BASF, Parsippany, N.J.), and phosphate buffered saline (PBS).

Administering may include delivering a dose of 1 ng/kg/day to 100 μg/kg/day of the fusion protein or conjugate product. The dose may be any value between 1 ng/kg/day to 100 μg/kg/day. The dose may be any dose between and including any two integer values between 1 ng/kg/day to 100 μg/kg/day. The dose may be 10, 15, 20, 25, 30, 35, 40, 45, 50, 55, 60, 65, 70, 75, 80, 85, 90, 95, or 100 ng/kg/day or mg/kg/day or any dose in a range between any two of the foregoing. Preferably, the dose may be about 16 ng/kg/day. Administering may include delivering any dose of a complementing therapeutic. The complementing therapeutic dose may be any 1 to 100 mg/kg/day. The complementing therapeutic dose may be any value between 1 to 100 mg/kg/day. The complementing therapeutic dose may be any dose between and including any two integer values between 1 ng/kg/day to 100 mg/kg/day. The complementing therapeutic dose may be 10, 15, 20, 25, 30, 35, 40, 45, 50, 55, 60, 65, 70, 75, 80, 85, 90, 95, or 100 mg/kg/day or any dose in a range between any two of the foregoing. The complementing therapeutic may be any one or more of nanoparticle (e.g. gold nanoparticles, liposomes), a therapeutic agent (e.g. cytokines, chemotherapeutic drugs, antibodies and antibody fragments, toxins, nucleic acids), a diagnostic agent (e.g. radioactive compounds, fluorescence compounds, chemiluminescent compounds), a contrasting agent (e.g. microbubbles), or cellular components (e.g. chimeric antigen receptors or TCRs). The concentration of the peptide(s) and at least one complementing therapeutic in the composition may be set to deliver the daily (or weekly, every three weeks or monthly) dosage in a single administration, two-point administrations, multiple point administrations, or continuous administration (e.g., intravenously, transdermally, intraperitoneally, by isolated limb perfusion, by isolated hepatic perfusion, or local administration) over a period of time. The period may be one day. The period may be 1, 2, 4, 8, 12, or 24 hours or a time within a range between any two of these values. The peptide-saporin and complementing therapeutic can be administered simultaneously or with 1, 2, 4, 8, 12, 24, 48 hours of delay or anticipation or any intermediate times.

A composition including fusion protein or conjugate product of the invention may include any amount of the protein or product. The amount may be that sufficient to deliver the dosage as set forth above in a suitable volume or sized delivery mode. When the dosage is split into multiple administrations throughout a time period, the amount in one volume or delivery mode may be the total dosage divided by the number of administrations throughout the time period. When present in a composition, the complementing therapeutic may be at any complementing therapeutic amount. Like the peptide, the complementing therapeutic amount may be tailored to deliver the right complementing therapeutic amount in the volume or delivery mode used for administration. The patient may be an animal. The patient may be a mammal. The patient may be a human. The patient may be a cancer patient. The cancer patient may be a lymphoma, or sarcoma, melanoma oral or skin squamous cell carcinoma, hepatocellular carcinoma, head and neck, gastroesophageal, colorectal, pancreatic, ovarian, lung, cervix, breast cancer, renal, urothelial, brain tumors (e.g. glioblastoma and astrocytoma) cancer patient, or patients with other solid-tumors or with metastasis of said tumors. The route for administering a composition or pharmaceutical composition may be by any route. The route of administration may be any one or more route including but not limited to oral, injection, topical, enteral, rectal, gastrointestinal, sublingual, sublabial, buccal, epidural, intracerebral, intracerebroventricular, intracisternal, epicutaneous, intraderm al, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transdermal, intratumoral, and transmucosal. Embodiments include a method of making the peptides of the invention, including the stapled peptide.

The method may include synthesizing a fusion protein or conjugate product having the sequence of the selected modified peptide.

The method may include evaluating the binding to CD13 of the peptide or to the other herein mentioned targets. Methods and conditions for evaluating the binding of the peptide may be set forth in the Example below.

An embodiment includes fusion protein or conjugate product or a composition thereof comprising a peptide consisting of, consisting essentially of, or comprising the sequence of any amino acid sequence herein. The peptide composition may include any complementing therapeutic herein. The peptide composition may include a pharmaceutically acceptable carrier.

The term “protein” includes single-chain polypeptide molecules as well as multiple-polypeptide complexes where individual constituent polypeptides are linked by covalent or non-covalent means. The term “polypeptide” includes peptides of two or more amino acids in length, typically having more than 5, 10 or 20 amino acids.

It will be understood that polypeptide sequences or peptides or proteins of the invention are not limited to the particular sequences or fragments thereof but also include homologous sequences obtained from any source, for example related viral bacterial proteins, cellular homologues and synthetic peptides, as well as variants or derivatives thereof. Polypeptide sequences of the present invention also include polypeptides encoded by polynucleotides of the present invention.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence preferably has targeting activity, preferably having at least 25 to 50% of the activity as the polypeptides herein presented, more preferably at least substantially the same activity. Thus, sequences may be modified for use in the present invention. Typically, modifications are made that maintain the activity of the sequence. Thus, in one embodiment, amino acid substitutions may be made, for example from 1, 2 or 3 to 10, 20 or 30 substitutions provided that the modified sequence retains at least about 25 to 50% of, or substantially the same activity. However, in an alternative embodiment, modifications to the amino acid sequences of a polypeptide of the invention may be made intentionally to reduce the biological activity of the polypeptide. For example, truncated polypeptides that remain capable of binding to target molecule but lack functional effector domains may be useful. In general, preferably less than 20%, 10% or 5% of the amino acid residues of a variant or derivative are altered as compared with the corresponding region depicted in the sequence listings. Amino acid substitutions may include the use of non-naturally occurring analogues, for example to increase blood plasma half-life of a therapeutically administered polypeptide.

Polypeptides of the invention also include fragments of the above-mentioned polypeptides and variants thereof, including fragments of the sequences. Preferred fragments include those which include an epitope. Suitable fragments will be at least about 5, e.g. 10, 12, 15 or 20 amino acids in length. They may also be less than 200, 100 or 50 amino acids in length. Polypeptide fragments of the proteins and allelic and species variants thereof may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues depicted in the sequence listings are altered. Proteins of the invention are typically made by recombinant means. However, they may also be made by synthetic means using techniques well known to skilled persons such as solid phase synthesis. Various techniques for chemical synthesising peptides are reviewed by Borgia and Fields, 2000, TibTech 18: 243-251 and described in detail in the references contained therein. Methods for preparing the conjugates of the invention have been described e.g. in WO01/61017. For instance, saporin can be fused with the CNGRCG (SEQ ID NO: 4) peptide by genetic engineering or by chemical synthesis.

The present invention will be described by means of non-limiting examples, referring to the following figures:

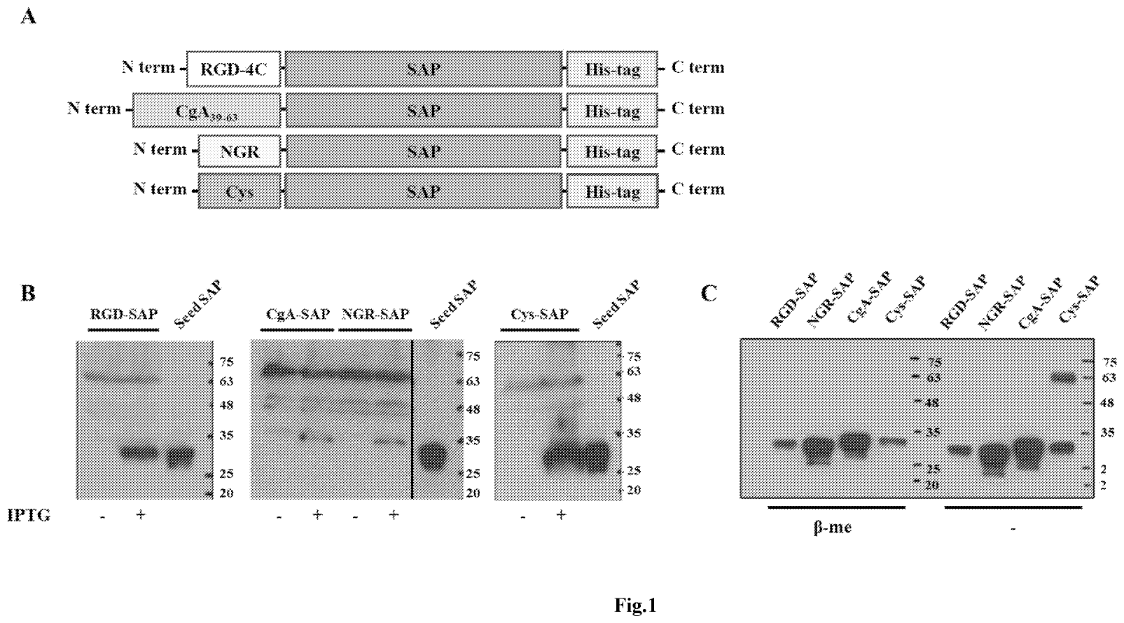

FIG. 1 Design, expression and purification of NGR-SAP, RGD-SAP, CgA-SAP and CYS-SAP recombinant proteins. (A) Schematic representation of SAP-based recombinant proteins. The NGR motif, RGD-4C, CgA39-63 targeting and CYS (CGGSGG) (SEQ ID NO:39) non-targeting peptides were inserted at the N-terminus of SAP. A stretch of six histidines (His)-tag was added at the C-terminus of the constructs. (B) Effect of IPTG on NGR-SAP, RGD-SAP, CgA-SAP and CYS-SAP expression in BL21DE3 E. coli cells as determined by western blot using an anti-SAP antibody. IPTG (+) and (−), induced and not induced cells, respectively. Seed-derived SAP (200 ng) was used as positive control for WB analysis. (C) WB analysis of purified NGR-SAP, RGD-SAP, CgA-SAP and CYS-SAP under reducing (β-mercaproethanol—β-me) or non-reducing (−) conditions.

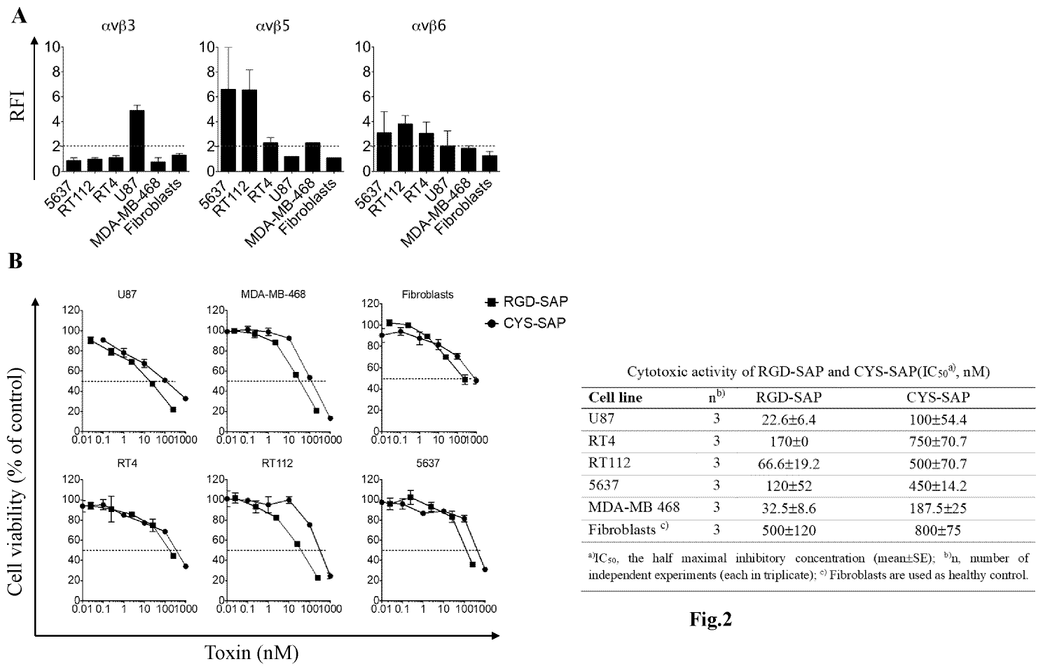

FIG. 2 Integrins expression on cancer cell lines and in vitro biological activity evaluation of RGD-SAP and CYS-SAP recombinant proteins. (A) 5637, RT112, RT4, U87, MDA-MB-468 cancer cell lines and fibroblasts were analyzed by flow cytometry for αvβ3, αvβ5 and αvβ6 integrins expression. Relative Fluorescence Intensity (RFI) are shown as mean±SD. The dashed line represents the threshold arbitrarily defining positive expression (RFI=2). (B) Effect of various amounts of RGD-SAP or CYS-SAP on the indicated cells as determined by MTT assay after 72 h treatment. One representative experiment out of three performed is shown. Cell viability is expressed as percentage to untreated cells (mean±SD). The IC₅₀ from three different experiments is reported as mean±SE.

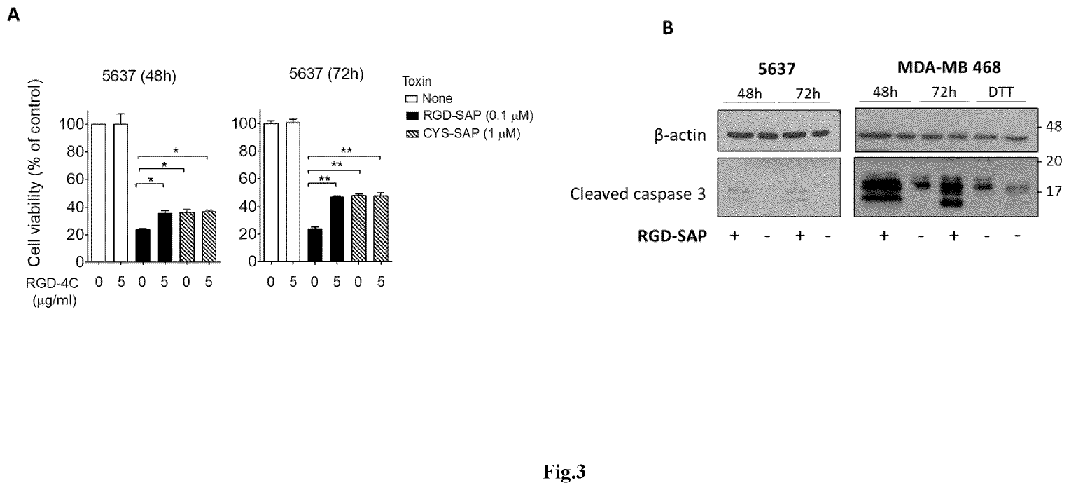

FIG. 3 RGD-SAP target specificity and caspase 3 activation. (A) 5637 bladder cancer cells were incubated with RGD-SAP (0.1 μM) or CYS-SAP (1 μM) in the presence or absence of ACDCRGDCFC peptide (5 μM). The cell viability was evaluated after 48 and 72 h by MTT assay. Results are shown as mean±SE. *p<0.05, **p<0.01 by non-parametric unpaired t test. (B) The activation of apoptosis via caspase 3 cleavage upon RGD-SAP (30 nM) treatment was evaluated in 5637 and MDA-MB-468 cells by analyzing the intracellular levels of caspase-3. Cells were incubated with RGD-SAP for 48 and 72 h and cell lysates were analyzed by western blot with anti-caspase-3 or an anti-3-actin antibodies for protein normalization. DTT (2 mM) was included as positive control for caspase-3 activation.

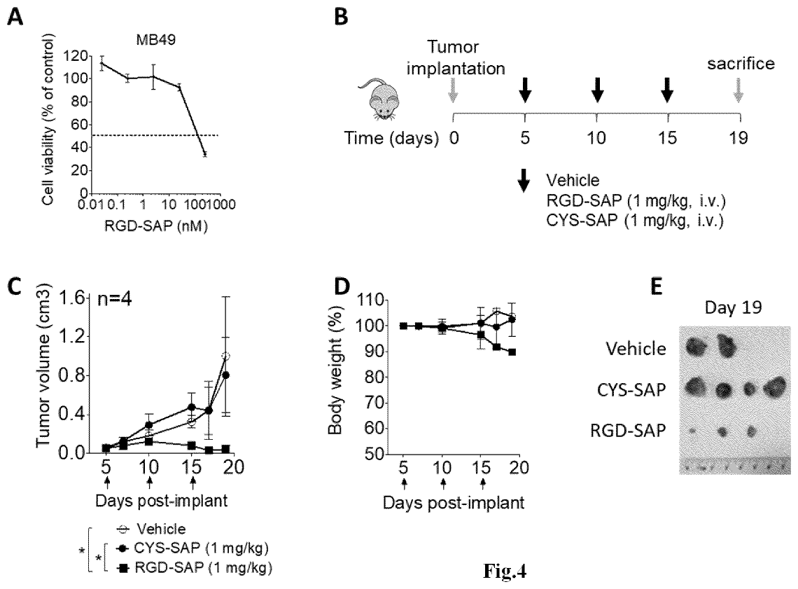

FIG. 4 Effects of RGD-SAP and CYS-SAP on tumor growth in a subcutaneous syngeneic bladder cancer mouse model. (A) Effect of RGD-SAP on MB49 murine bladder cancer cell line viability as measured by MTT assay. (B) Schematic representation of the experimental design and treatment schedule. MB49 cells were subcutaneously implanted in 7-week-old C57BL/6 WT female mice. Tumor bearing-mice were treated i.v. with the indicated doses of CYS-SAP or RGD-SAP or vehicle every 5 days after tumor implantation (arrows). (C) Effect of 1 mg/kg dose of CYS-SAP or RGD-SAP on MB49 bladder cancer growth. Tumor volumes (mean±SD, n=4 mice/group) are shown. *p<0.05, by one-way ANOVA test. (D) Effect of treatments on the animal body weight change. Animal weights are reported as percent of starting body weight (mean±SD of 4 animals per group). (E) A representative image of MB49 subcutaneous tumors explanted from mice that survived to the end of the experiment (day 19): (vehicle (n=2), 2 mice were sacrificed at day 16 due to tumor ulceration; RGD-SAP (n=4), 1 tumor undetectable).

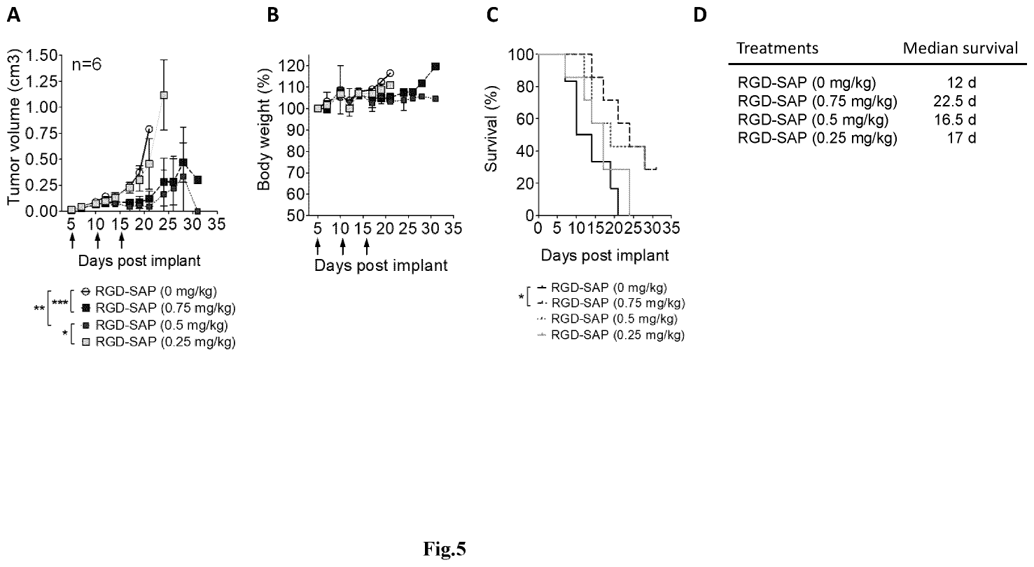

FIG. 5 Dose dependent effects of RGD-SAP on tumor growth in a subcutaneous syngeneic bladder cancer mouse model. (A) MB49 cells were subcutaneously implanted in 7-week-old C57BL/6 WT female mice (n=6/group) were treated i.v. with the indicated doses of RGD-SAP every 5 days after tumor implantation for three times (arrows). Tumor volumes (mean±SD) are shown. *p<0.05; **p<0.01; ***p<0.001 by using one-way ANOVA test. (B) Animal weights are reported as percent of starting body weight (mean±SD of 6 animals per group). (C) Kaplan-Meyer plot of animal survival and (D) the associated median survival time. Results from a Mantel-Cox two-sided log-rank test are shown when statistically significant (*p<0.05) for RGD-SAP 0.75 mg/kg (dashed line, hazard ratio 5.25; 95% CI, 1.19-23.10) versus vehicle (black line).

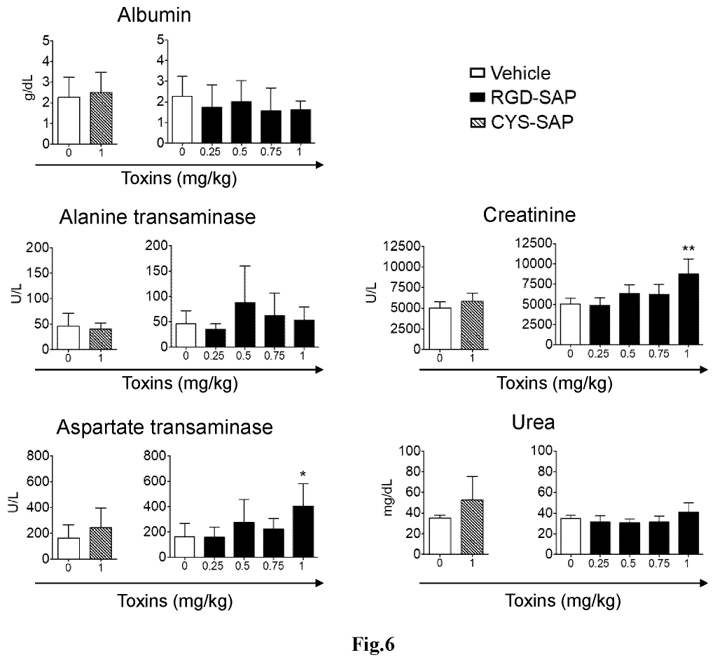

FIG. 6 Comparison of clinical biochemistry parameters in mice treated with CYS-SAP or different RGD-SAP concentrations. Blood samples were collected from treated mice and the concentration of albumin, alanine transaminase, aspartate transaminase, creatinine and urea in the serum were measured. Data are shown as mean±SD from n=4/6 mice per condition. Results from unpaired Student t test are shown. All treatments were compared to vehicle (*p<0.05; **p<0.01).

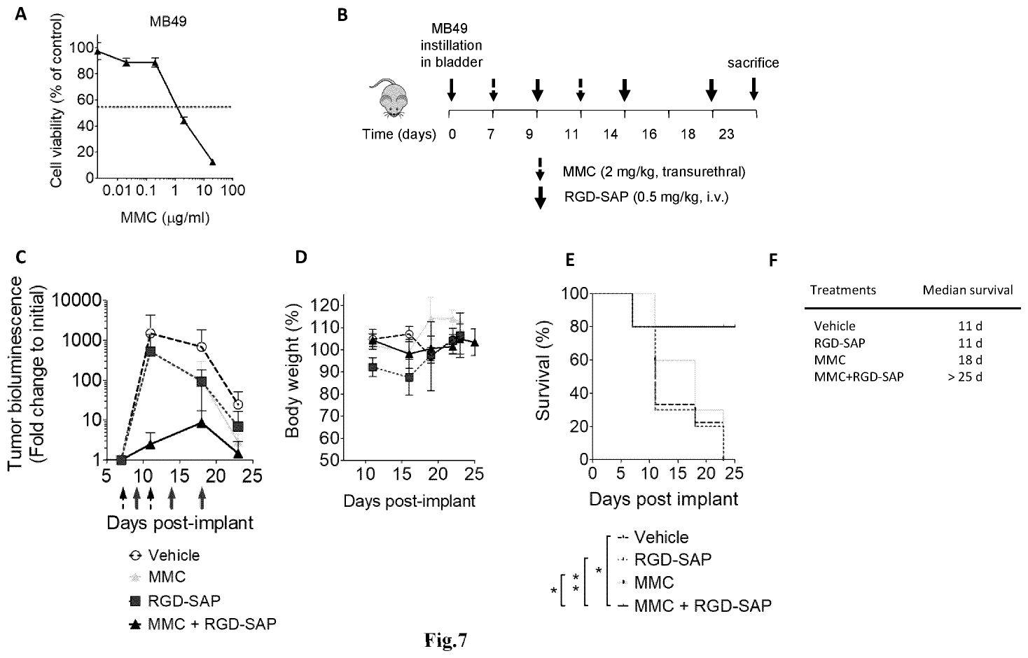

FIG. 7 Effects of MMC and RGD-SAP on tumor growth in an orthotopic syngeneic bladder cancer mouse model. (A) Effect of mitomycin C (MMC) on MB49 murine bladder cancer cell line viability as measured by MTT assay. (B) Schematic representation of the in vivo experimental design and treatment schedule. Luciferase-expressing MB49 cells were transurethrally injected into the bladder of 7-week-old C57BL/6 female mice. Tumor engraftment was monitored by in vivo bioluminescence imaging. Tumor bearing mice were randomized into 4 experimental groups and treated with the indicated dose of MMC (transurethral, dashed arrows, n=10 mice), RGD-SAP (i.v., bold arrows, n=10 mice), MMC+RGD-SAP (n=5 mice) or vehicle (n=10 mice). The results of two experiments with 5 animals/group were considered and cumulated (C) Tumor growth (mean±SD) as determined over time by fold change to the initial detectable bioluminescence. (D) Effect of treatments on the animal body weight change. Animal weights are reported as percent of starting body weight (mean±SD of 5-10 animals per group). (E) Kaplan-Meyer plot of animal survival and (F) median survival time. Results from a Mantel-Cox two-sided log-rank test are shown when statistically significant (*p<0.05, **p<0.01) for MMC+RGD-SAP (black line) versus vehicle (dashed line, hazard ratio 7.19; 95% CI, 1.51-34.26) or MMC (grey line, hazard ratio 5.96, 95% CI, 1.44-24.69) or RGD-SAP (dotted line, hazard ratio 0.15, 95% CI, 0.03-0.62).

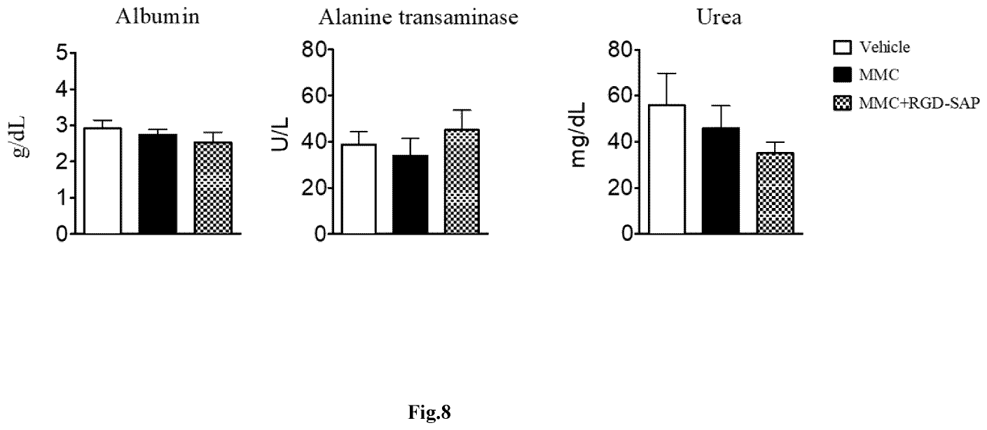

FIG. 8 Comparison of clinical biochemistry parameters in mice treated with mitomycin C (MMC) alone or in combination with RGD-SAP. Blood samples were collected from treated mice and the concentration of albumin, alanine transaminase and urea in the serum were measured. Values are shown as mean±SD from n=10 for vehicle and MMC; n=5 for MMC+RGD-SAP.

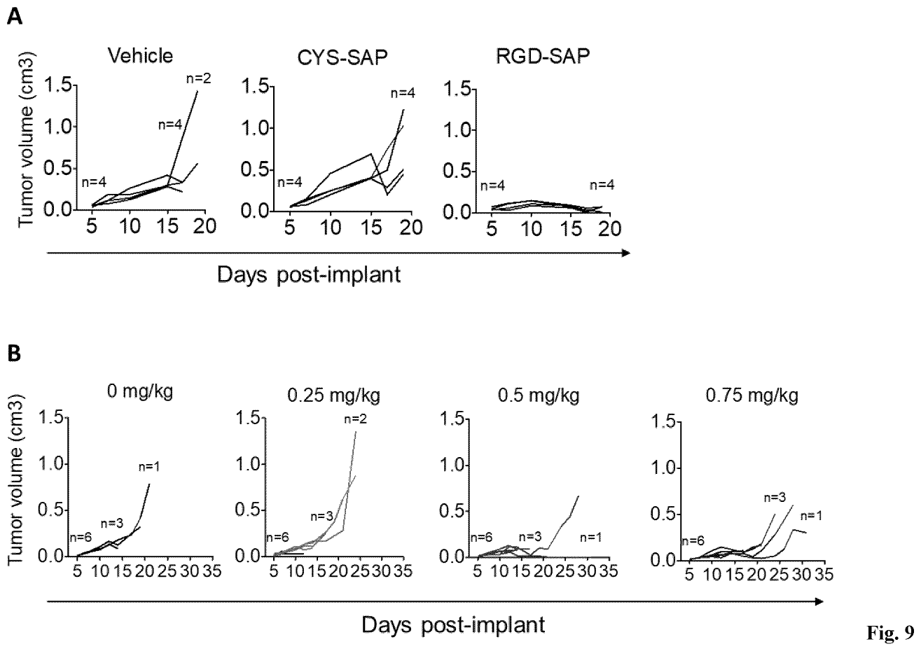

FIG. 9 (A) Quantitative analysis of growing tumor volume for each individual mouse treated with vehicle, RGD-SAP or CYS-SAP. (B) Quantitative analysis of growing tumor volume for each individual mouse treated with vehicle (0 mg/kg) or RGD-SAP 0.25, 0.5 and 0.75 mg/kg).

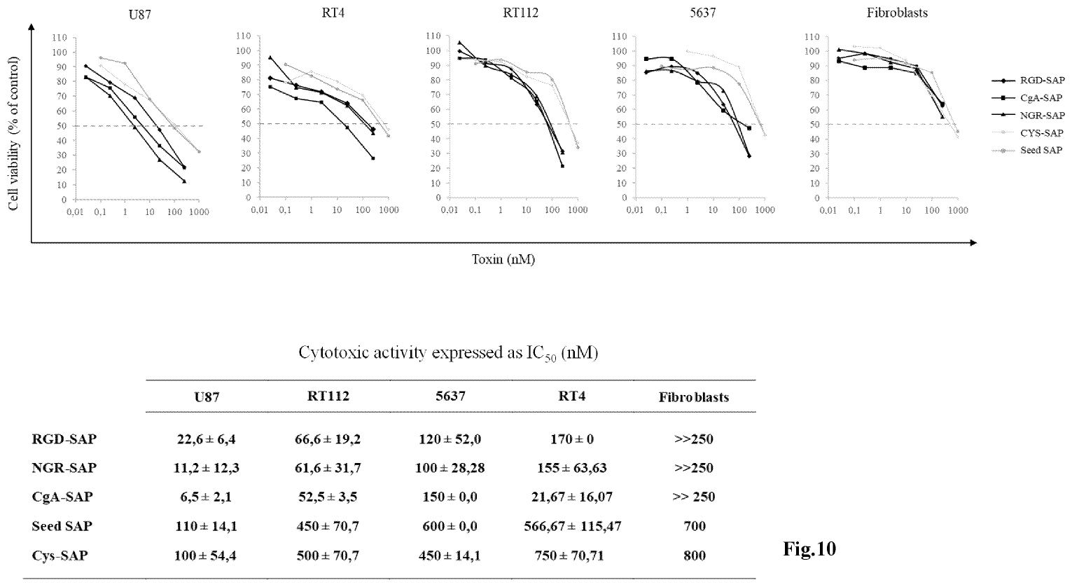

FIG. 10 Biological activity evaluation of NGR-SAP, RGD-SAP and CgA-SAP recombinant proteins. Effects of various amounts of NGR-SAP, RGD-SAP and CgA-SAP on 5637, RT112, RT4, U87 cancer cell lines and fibroblasts as determined by MTT assay after 72 h treatment. Recombinant CYS-SAP and seed-derived SAP were used as untargeted controls. One representative experiment out of three performed is shown. Cell viability is expressed as percentage to untreated cells (mean±SD). The IC₅₀ from three different experiments is reported as mean SE.

### SEQUENCES

Starting from the N-terminus, the recombinant proteins of the invention shown below comprises a targeting domain (RGD-4C, NGR motif or CgA39-63) followed by a linker (GGSSRSS, underlined in the sequences below), the sequence of mature saporin (in bold in the sequence below) and a 6-histine tail. The VDK sequence (shown in italic below) has the functions of increasing the distance between saporin and the histidine tail and of facilitating the recognition of the latter in case of affinity purification. Further, since the DNA sequence coding for the VD amino acids contains a SalI restriction site (GTCGAC) and another site is located in the pEt22b plasmid where the sequences were cloned, just after EcoRI used for cloning, by cutting the plasmid with SalI, it is possible to remove the coding sequence of the histidine tail and easily replace it with another tag (obtaining a versatile platform) or to close the plasmid using its natural histidine tail which is 6 amino acids further away from saporin, one more chance in case there were difficulties in the purification process, but this is not the case.

RGD-SAP-His
(SEQ ID NO: 6)
ACDCRGDCFCGGGSSRSSVTSITLDLVNPTAGQYSSFVDKIRNNVKDPNLKYGGTDIA
VIGPPSKEKFLRINFQSSRGTVSLGLKRDNLYVVAYLAMDNTNVNRAYYFKSEITSA
ELTALFPEATTANQKALEYTEDYQSIEKNAQITQGDKSRKELGLGIDLLLTFMEAV
NKKARVVKNEARFLLIAIQMTAEVARFRYIQNLVTKNFPNKFDSDNKVIQFEVSWR
KISTAIYGDAKNGVFNKDYDFGFGKVRQVKDLQMGLLMYLGKPKVDKHHHHHH
NGR-SAP-His
(SEQ ID NO: 7)
CNGRCGGGSSRSSVTSITLDLVNPTAGQYSSFVDKIRNNVKDPNLKYGGTDIAVIGPP
SKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKA
RVVKNEARFLLIAIQMTAEVARFRYIQNLVTKNFPNKFDSDNKVIQFEVSWRKISTA
IYGDAKNGVFNKDYDFGFGKVRQVKDLQMGLLMYLGKPKVDKHHHHHH
CgA₃₉₋₆₃-SAP-His
(SEQ ID NO: 8)
FETLRGDERILSILRHQNLLKELQDGGSSRSSVTSITLDLVNPTAGQYSSFVDKIRNNVK
DPNLKYGGTDIAVIGPPSKEKFLRINFQSSRGTVSLGLKRDNLYVVAYLAMDNTNV
NRAYYFKSEITSAELTALFPEATTANQKALEYTEDYQSIEKNAQITQGDKSRKELGL
GIDLLLTFMEAVNKKARVVKNEARFLLIAIQMTAEVARFRYIQNLVTKNFPNKFDS
DNKVIQFEVSWRKISTAIYGDAKNGVFNKDYDFGFGKVRQVKDLQMGLLMYLGK
PKVDKHHHHHH

The nucleotide sequence coding for SEQ ID NO:6 is:

(SEQ ID NO: 9)
GCTTGTGATTGTAGGGGAGATTGTTTTTGTGGAGGGGGATCcAGTAGAT
CAAGCGTGACCAGCATCACCCTGGACCTGGTTAACCCGACCGCGGGCCA
GTACAGCAGCTTTGTGGACAAGATTCGTAACAACGTTAAGGACCCGAAC
CTGAAATATGGCGGTACCGATATCGCGGTGATTGGTCCGCCGAGCAAGG
AGAAATTCCTGCGTATCAACTTTCAAAGCAGCCGTGGCACCGTTAGCCT
GGGTCTGAAGCGTGACAACCTGTACGTGGTTGCGTATCTGGCGATGGAT
AACACCAACGTGAACCGTGCGTACTATTTCAAAAGCGAGATTACCAGCG
CGGAACTGACCGCGCTGTTTCCGGAAGCGACCACCGCGAACCAGAAGGC
GCTGGAGTACACCGAAGACTATCAAAGCATCGAGAAAAACGCGCAGATT
ACCCAAGGTGACAAGAGCCGTAAAGAACTGGGCCTGGGTATCGATCTGC
TGCTGACCTTCATGGAGGCGGTTAACAAGAAAGCGCGTGTGGTTAAGAA
CGAAGCGCGTTTCCTGCTGATCGCGATTCAGATGACCGCGGAAGTGGCG
CGTTTTCGTTACATCCAAAACCTGGTTACCAAGAACTTCCCGAACAAAT
TTGACAGCGATAACAAAGTGATTCAGTTCGAAGTTAGCTGGCGTAAAAT
CAGCACCGCGATTTACGGCGATGCGAAGAACGGTGTGTTTAACAAAGAC
TATGATTTCGGCTTTGGCAAAGTGCGTCAGGTTAAAGACCTGCAAATGG
GCCTGCTGATGTATCTGGGCAAGCCGAAAgtcgacAAACACCACCACCA
CCACCAC

The nucleotide sequence coding for SEQ ID NO:7 is:

(SEQ ID NO: 10)
TGTAATGGAAGGTGTGGAGGGGGATCcAGTAGATCAAGTGTAACATCAA
TAACCCTGGACCTGGTTAACCCGACCGCGGGCCAGTACAGCAGCTTCGT
GGACAAGATTCGTAACAACGTTAAGGACCCGAACCTGAAATATGGCGGT
ACCGATATCGCGGTGATTGGTCCGCCGAGCAAGGAGAAATTCCTGCGTA
TCAACTTTCAAAGCAGCCGTGGCACCGTTAGCCTGGGTCTGAAGCGTGA
CAACCTGTACGTGGTTGCGTATCTGGCGATGGATAACACCAACGTGAAC
CGTGCGTACTATTTCAAAAGCGAGATTACCAGCGCGGAACTGACCGCGC
TGTTTCCGGAAGCGACCACCGCGAACCAGAAGGCGCTGGAGTACACCGA
AGACTATCAAAGCATCGAGAAAAACGCGCAGATTACCCAAGGTGACAAG
AGCCGTAAAGAACTGGGCCTGGGTATCGATCTGCTGCTGACCTTTATGG
AGGCGGTTAACAAGAAAGCGCGTGTGGTTAAGAACGAAGCGCGTTTCCT
GCTGATCGCGATTCAGATGACCGCGGAAGTGGCGCGTTTTCGTTACATC
CAAAACCTGGTTACCAAGAACTTCCCGAACAAATTTGACAGCGATAACA
AAGTGATTCAGTTCGAAGTTAGCTGGCGTAAAATCAGCACCGCGATTTA
CGGCGATGCGAAGAACGGTGTGTTTAACAAAGACTATGATTTCGGCTTT
GGCAAAGTGCGTCAGGTTAAAGACCTGCAAATGGGCCTGCTGATGTATC
TGGGCAAGCCGAAAgtcgacAAACACCACCACCACCACCAC

The nucleotide sequence coding for SEQ ID NO:8 is:

(SEQ ID NO: 11)
TTTGAAACACTAAGGGGAGATGAAAGAATATTATCAATACTAAGGCACC
AAAACCTGCTGAAGGAGCTGCAAGACGGTGGatcCAGCCGTAGCAGCGT
GACCAGCATCACCCTGGATCTGGTTAACCCGACCGCGGGCCAGTACAGC
AGCTTTGTGGACAAAATTCGTAACAACGTTAAGGACCCGAACCTGAAAT
ATGGTGGCACCGATATCGCGGTGATTGGTCCGCCGAGCAAGGAGAAATT
CCTGCGTATCAACTTTCAAAGCAGCCGTGGTACCGTTAGCCTGGGCCTG
AAGCGTGACAACCTGTACGTGGTTGCGTATCTGGCGATGGATAACACCA
ACGTGAACCGTGCGTACTATTTCAAAAGCGAGATTACCAGCGCGGAACT
GACCGCGCTGTTTCCGGAAGCGACCACCGCGAACCAGAAGGCGCTGGAG
TACACCGAAGACTATCAAAGCATCGAGAAAAACGCGCAGATTACCCAAG
GCGACAAGAGCCGTAAAGAACTGGGTCTGGGCATCGATCTGCTGCTGAC
CTTCATGGAGGCGGTTAACAAGAAAGCGCGTGTGGTTAAGAACGAAGCG
CGTTTCCTGCTGATCGCGATTCAGATGACCGCGGAAGTGGCGCGTTTTC
GTTACATCCAAAACCTGGTTACCAAGAACTTCCCGAACAAATTTGACAG
CGATAACAAAGTGATTCAGTTCGAAGTTAGCTGGCGTAAAATCAGCACC
GCGATTTACGGTGATGCGAAGAACGGCGTGTTTAACAAAGACTATGATT
TCGGTTTTGGCAAAGTGCGTCAGGTTAAAGACCTGCAAATGGGTCTGCT
GATGTATCTGGGCAAGCCGAAAgtcgacAAACACCACCACCACCACCAC

In the following sequences the bases before and after the fusion peptide coding sequence are restriction sites for cloning and start signal (ATG) and end signal (TAA)

>RGD-SAP-His_opt
(SEQ ID NO: 20)
catATGGCTTGTGATTGTAGGGGAGATTGTTTTTGTGGAGGGGGATCcAGTAGATCAA
GCGTGACCAGCATCACCCTGGACCTGGTTAACCCGACCGCGGGCCAGTACAGC
AGCTTTGTGGACAAGATTCGTAACAACGTTAAGGACCCGAACCTGAAATATGG
CGGTACCGATATCGCGGTGATTGGTCCGCCGAGCAAGGAGAAATTCCTGCGTA
TCAACTTTCAAAGCAGCCGTGGCACCGTTAGCCTGGGTCTGAAGCGTGACAAC
CTGTACGTGGTTGCGTATCTGGCGATGGATAACACCAACGTGAACCGTGCGTA
CTATTTCAAAAGCGAGATTACCAGCGCGGAACTGACCGCGCTGTTTCCGGAAG
CGACCACCGCGAACCAGAAGGCGCTGGAGTACACCGAAGACTATCAAAGCATC
GAGAAAAACGCGCAGATTACCCAAGGTGACAAGAGCCGTAAAGAACTGGGCCT
GGGTATCGATCTGCTGCTGACCTTCATGGAGGCGGTTAACAAGAAAGCGCGTG
TGGTTAAGAACGAAGCGCGTTTCCTGCTGATCGCGATTCAGATGACCGCGGAA
GTGGCGCGTTTTCGTTACATCCAAAACCTGGTTACCAAGAACTTCCCGAACAAA
TTTGACAGCGATAACAAAGTGATTCAGTTCGAAGTTAGCTGGCGTAAAATCAGC
ACCGCGATTTACGGCGATGCGAAGAACGGTGTGTTTAACAAAGACTATGATTT
CGGCTTTGGCAAAGTGCGTCAGGTTAAAGACCTGCAAATGGGCCTGCTGATGT
ATCTGGGCAAGCCGAAAgtcgacAAACACCACCACCACCACCACTAAGAATTC
>NGR-SAP-His_opt
(SEQ ID NO: 21)
catATGTGTAATGGAAGGTGTGGAGGGGGATCcAGTAGATCAAGTGTAACATCAATA
ACCCTGGACCTGGTTAACCCGACCGCGGGCCAGTACAGCAGCTTCGTGGACAA
GATTCGTAACAACGTTAAGGACCCGAACCTGAAATATGGCGGTACCGATATCG
CGGTGATTGGTCCGCCGAGCAAGGAGAAATTCCTGCGTATCAACTTTCAAAGC
AGCCGTGGCACCGTTAGCCTGGGTCTGAAGCGTGACAACCTGTACGTGGTTGC
GTATCTGGCGATGGATAACACCAACGTGAACCGTGCGTACTATTTCAAAAGCG
AGATTACCAGCGCGGAACTGACCGCGCTGTTTCCGGAAGCGACCACCGCGAAC
CAGAAGGCGCTGGAGTACACCGAAGACTATCAAAGCATCGAGAAAAACGCGCA
GATTACCCAAGGTGACAAGAGCCGTAAAGAACTGGGCCTGGGTATCGATCTGC
TGCTGACCTTTATGGAGGCGGTTAACAAGAAAGCGCGTGTGGTTAAGAACGAA
GCGCGTTTCCTGCTGATCGCGATTCAGATGACCGCGGAAGTGGCGCGTTTTCG
TTACATCCAAAACCTGGTTACCAAGAACTTCCCGAACAAATTTGACAGCGATAA
CAAAGTGATTCAGTTCGAAGTTAGCTGGCGTAAAATCAGCACCGCGATTTACG
GCGATGCGAAGAACGGTGTGTTTAACAAAGACTATGATTTCGGCTTTGGCAAA
GTGCGTCAGGTTAAAGACCTGCAAATGGGCCTGCTGATGTATCTGGGCAAGCC
GAAAgtcgacAAACACCACCACCACCACCACTAAGAATTC
>CgA39-63-SAP-His_opt
(SEQ ID NO: 22)
catATGTTTGAAACACTAAGGGGAGATGAAAGAATATTATCAATACTAAGGCACCAA
AACCTGCTGAAGGAGCTGCAAGACGGTGGatcCAGCCGTAGCAGCGTGACCAGCAT
CACCCTGGATCTGGTTAACCCGACCGCGGGCCAGTACAGCAGCTTTGTGGACA
AAATTCGTAACAACGTTAAGGACCCGAACCTGAAATATGGTGGCACCGATATC
GCGGTGATTGGTCCGCCGAGCAAGGAGAAATTCCTGCGTATCAACTTTCAAAG
CAGCCGTGGTACCGTTAGCCTGGGCCTGAAGCGTGACAACCTGTACGTGGTTG
CGTATCTGGCGATGGATAACACCAACGTGAACCGTGCGTACTATTTCAAAAGC
GAGATTACCAGCGCGGAACTGACCGCGCTGTTTCCGGAAGCGACCACCGCGAA
CCAGAAGGCGCTGGAGTACACCGAAGACTATCAAAGCATCGAGAAAAACGCGC
AGATTACCCAAGGCGACAAGAGCCGTAAAGAACTGGGTCTGGGCATCGATCTG
CTGCTGACCTTCATGGAGGCGGTTAACAAGAAAGCGCGTGTGGTTAAGAACGA
AGCGCGTTTCCTGCTGATCGCGATTCAGATGACCGCGGAAGTGGCGCGTTTTC
GTTACATCCAAAACCTGGTTACCAAGAACTTCCCGAACAAATTTGACAGCGATA
ACAAAGTGATTCAGTTCGAAGTTAGCTGGCGTAAAATCAGCACCGCGATTTAC
GGTGATGCGAAGAACGGCGTGTTTAACAAAGACTATGATTTCGGTTTTGGCAA
AGTGCGTCAGGTTAAAGACCTGCAAATGGGTCTGCTGATGTATCTGGGCAAGC
CGAAAgtcgacAAACACCACCACCACCACCACTAAGAATTC

### EXAMPLE 1

### Materials and Methods

### Cell Cultures

Human bladder RT4, RT112, 5637 were maintained in RPMI 1640 supplemented with 10% fetal calf serum (FCS), 2 mM L-glutamine and antibiotics (100 U/mL penicillin and 100 μg/mL streptomycine-sulphate); breast MDA-MB 468 and glioblastoma U87 cancer cell lines as well as skin fibroblast cells were maintained in DMEM supplemented with 10% FCS, 2 mM L-glutamine and antibiotics (100 U/mL penicillin and 100 μg/mL streptomycine-sulphate). Murine MB49 bladder cancer cells were cultured in DMEM, supplemented with 10% FCS, 2 mM L-glutamine, antibiotics (100 U/mL penicillin and 100 μg/mL streptomycine-sulphate) and 1 mM sodium pyruvate.

MB49 Luc cells stably expressing luciferase were generated by transduction with a 3^{rd} generation lentiviral vector carrying the luciferase gene. pLenti PGK V5-LUC Neo (w623-2) was a gift from Eric Campeau, University of Massachusetts Medical School, Worcester, Massachusetts, US (Addgene plasmid #21471). For lentivirus production, a monolayer of HEK293T cells, cultured in 10 cm² dish, were incubated with the following mixture: transfer vector (10 μg), packaging vector Δr 8.74 (6.5 μg), Env VSV-G vector (3.5 μg), REV vector (2.5 μg) in 450 μl double distilled water, 50 μl calcium chloride (2.5 M) and 500 μl Hank's buffered saline (2-fold). Sixteen hours later, the medium was replaced with culture medium and 24 hours later the medium was collected and 0.22 μm-filtered to recover virus particles. Virus particles were then used to transduce MB49 cells. Infected cells were then cultured in presence of G418 antibiotic (0.5 mg/ml) for fifteen days.

Cloning of RGD-SAP and CYS-SAP in pET22b Vector

SAP fused with ACDCRGDCFCG or CGGSGG at its N-terminus were prepared by GenScript (New Jersey, USA). The nucleotide sequences were obtained from the corresponding amino acid sequences of saporin S and optimized for the expression in E. coli. A GGSSRSS sequence was interposed between ACDCRGDCFC and SAP as a spacer and a 6×His tag was added at the C-terminus to allow the purification by affinity chromatography. The whole encoding sequences were inserted into the pET22b(+) vector (Novagen), forming the pET22b(+)-RGD-SAP (5′-NdeI-ACDCRGDCFCG-GGSSRSS-SAP-H IHHH-EcoRI-3′) and the pET22b(+)-CYS-SAP (5′-NdeI-CGGSGG-SAP-H IHHH-EcoRI-3′) expression vectors. Ligation products were used to transformed the E. coli strain DH5alpha (Invitrogen).

### Expression and Purification of RGD-SAP and CYS-SAP

The expression of RGD-SAP and CYS-SAP in transformed BL21(DE3) E. coli cells (Novagen) was induced for 3 hours at 37° C. with 0.1 mM IPTG. Bacterial pellet from 1 L culture was resuspended in 15 ml of 50 mM Tris-HCl, pH 7.5, supplemented with a cocktail of protease inhibitor (Sigma-Aldrich). Soon after, 10 mM of imidazole, lysozyme (250 μg/ml) and DNAse (20 μg/ml) were added. The bacterial solution was then incubated on ice for 45 min, subjected to 3 cycles of sonication (using a UW3100 Bandelin sonicator operating at 60% power; 2 min cycle duration with 1 sec pulse and 1 sec pause), and centrifuged at 4° C. for 25 min at 10000×g. Soluble RGD-SAP and CYS-SAP contained in the supernatant were then purified by metal chelate affinity chromatography using a HisTrap HP 5 ml column (GE Healthcare Life Sciences) equilibrated in Tris-HCl 50 mM pH 7.5, 300 mM NaCl supplemented with 10 mM imidazole (and 5 mM DTT for CYS-SAP) operated with the AktaPurifier10 FPLC system. To elute proteins, imidazole concentration was increased step by step up to 500 mM in 20 column volumes (CV). The fractions containing the target proteins were dialyzed against 50 mM Tris-HCl, pH 7.5 (CYS-SAP) or 50 mM bicine, pH 8.2 (RGD-SAP) at 4° C. for 16 hours. A cation exchange chromatography on HiTrap SP Sepharose FF column (GE Healthcare Life Sciences) was then performed for further purification, using a 20 CV gradient up to 1 M NaCl for protein elution. The proteins were then concentrated using 10 KDa cutoff Amicon centrifugal filters (Millipore-Sigma) and dialyzed against PBS with slide A lyzer dialysis cassette (Thermo Fisher). All solutions used in purification steps were prepared with sterile and endotoxin-free water (S.A.L.F. Laboratorio Farmacologico SpA, Bergamo, Italy). Protein concentration was measured using the BCA Protein Assay DC™ Kit (BioRad). Protein purity and identity was checked by SDS-PAGE and Western blotting. Both CYS-SAP and RGD-SAP showed comparable yields ranging from 0.6 to 1.2 mg/l of bacterial culture.

### Western Blot Analysis

Cells were washed twice with cold PBS, collected by scraping and centrifuged 5 min at 300 g. Cells were lysed for 30 min on ice in ice-cold buffer (50 mM Tris-HCl, pH 7.5, containing 150 mM NaCl, 2 mM NaF, 1 mM EDTA, 1 mM EGTA, 1 mM Na₃VO₄, 1 mM PMSF, 75 mU/ml aprotinin (Sigma-Aldrich), 1% TritonX-100 and a proteinase inhibitor Cocktail® (Sigma-Aldrich). Cell lysates were centrifuged at 10000×g at 4° C. for 10 min and the supernatants were recovered and quantified for total protein content. Equal amounts of cell protein extracts were separated by SDS-PAGE under reducing conditions unless stated otherwise. For western blot analysis, proteins were transferred onto a nitrocellulose membrane, incubated with 5% non-fat powdered milk in TBS-T (0.5% Tween-20) for 1 hour and then with the following antibodies: anti-saporin anti-serum (rabbit, 1:5000), anti-caspase 3 (rabbit, 1:2000, clone E87, Abcam), anti-beta actin (mouse, 1:10000, clone AC-15, Sigma-Aldrich). The antibody binding was detected using a secondary horseradish peroxidase conjugated antibodies (donkey anti-mouse/rabbit IgG HRP-linked, GE Healthcare) and an enhanced chemiluminescent (ECL, Merck Millipore).

Seed SAP used as positive control for Western blot analysis was purchased from Advanced Targeting Systems, which had purified it from the seeds of the Soapwort plant (Saponaria officinalis).

### Flow Cytometry Analysis

Cultured cell lines were detached by TripLE Express (Gibco) to preserve receptor integrity, washed with PBS containing 1% FCS and incubated with PE-conjugated Ab specific for human αvβ3 and αvβ5 (R&D System) and FITC-conjugated Ab specific for human αvβ6 integrins (NovusBio). For receptor detection, cells were incubated with the fluorescently labelled Ab at 4° C. for 30 min. Stained cells were resuspended in 100 μL of PBS containing 1% FCS. Samples were run through an Accuri™ flow cytometer (BD Biosciences). All data were analysed by FCS Express and expressed as relative fluorescence intensity (RFI), calculated as follows: mean fluorescence intensity after mAb staining/mean fluorescence intensity after isotype-negative control staining. Analysis was done on 20,000 gated events acquired per sample.

### Cell Viability Assay (MTT)

Cultured cell lines (5×10³ cells/well) were seeded in 96 wells plates and incubated for 72 h with various amounts of RGD-SAP or CYS-SAP at 37° C., 5% CO₂. Cell viability was then quantified by 3-(4,5-dimethylthiazol-2-yl)-2,5-diphenyltetrazolium bromide staining (MTT) (working solution 0.5 μg/ml). After 1 h of incubation, the supernatants were removed, the formazan crystals were dissolved with dimethyl sulfoxide and the absorbance at 570 nm was measured using a microtiter plate reader. Competitive experiments were performed as described above using 100 nM of RGD-SAP or 1000 nM of CYS-SAP in the presence of 5000 nM of ACDCRGDCFCG peptide for 48 and 72 hours.

To induce caspase 3 activation, cells were treated with 30 nM RGD-SAP or 2 mM DTT for 48 and 72 hours.

### In Vivo Studies

Studies on animal models were approved by the Institutional Animal Care and Use Committee (Institutional Animal Care and Use Committee, IACUC) and performed according to the prescribed guidelines. C57BL/6 female mice (7 weeks old, Charles River, Calco, Italy) were challenged with subcutaneous injection in the left flank of 3-5×10⁵ MB49 living cells; 5 days later, 4-6 mice/group were intravenously administered with various doses of RGD-SAP or CYS-SAP diluted in sodium chloride (0.9%, i.v., 200 μl). Tumor growth was estimated by calculating the volume using the formula r₁×r₂×r₃×4/3 π, where r₁ and r₂ are the longitudinal and lateral radii, and r₃ is the thickness of tumors protruding from the surface of normal skin. Animals were euthanized when tumors reached 10 mm in diameter, became ulcerated or a 15% animal body weight loss was measured.

Orthotopic syngeneic tumor were developed according to the procedure described by Loskog et al. [27]. Briefly, C57BL/6 female mice were anesthetized with ketamine/xylazine (80/10 mg/kg) and catheterized (PE50 catheter, BD Biosciences) using lubricated catheters with 2.5% lidocaine-containing gel (Luan). To enhance tumor engraftment, 100 μl of poly-L-lysine (0.1 mg/ml, mw 70000-150000, Sigma Aldrich), was injected transurethrally into the bladder and left in place for 30 min, then bladder was washed with PBS and instilled with 5×10⁴ MB49 Luc diluted in serum-free medium (100 μl/mice), 30 min later the catheters were removed. On day 7, mice were treated with mitomycin C (MMC) alone (n=10), administered transurethrally and kept in the bladder for 1 hour (50 μg in 100 μl of PBS, every 4 days for 2 times), or combined with RGD-SAP (n=5) or RGD-SAP alone (i.v., 200 μl, every 5 days, for 3 times) (n=10), starting at day 9. Control group of mice was treated with vehicle (sodium chloride, 0.9%, i.v., 200 μl) (n=10). Orthotopic tumor engraftment and growth was monitored once a week by in vivo bioluminescence imaging (IVIS). Tumor growth was estimated by acquiring the bioluminescence signal (BLI) and it expressed in total photon flux. Mice were euthanized when the BLI intensity suddenly drop, due to irreversible necrosis and accompanied with hematuria or animal lethargy.

### Blood Sample Collection and Biochemical Parameters Analysis

Blood samples were collected from the retroorbital plexus of anesthetized mice using 4% isoflurane at the end of each experiment or before animal sacrifice. Blood samples were left at room temperature for at least 30 min before being processed and then centrifuged (800×g, 10 min) for serum separation. Serum albumin, aspartate transaminase, alanine transaminase, creatinine and urea were determined by using an automated analyzer (ScilVet ABC plus and Idexx Procyte analyzers) according to the manufacturers' instructions. Standard controls were run before each determination.

### Statistical Analysis

All in vitro experiments were performed at least in triplicate. Mouse experiments were performed using at least 4 mice per group. When appropriate, statistical significance was determined using a 2-tailed Student's t test. For tumor growth analyses, inventors performed one-way ANOVA statistical analysis. Survival curves were compared using the log rank test. Tests symbols mean: *p<0.05; **p<0.01; ***p<0.001; ns, not significant.

### Results

### Production and Characterization of RGD-SAP and CYS-SAP

RGD-SAP, consisting of RGD-4C fused to the N-terminus of SAP was produced in E. coli cells by recombinant DNA technology. In parallel inventors have also produced a SAP variant with a Cys residue in place of the RGD-4C domain (CYS-SAP) (FIGS. 1A and B).

To facilitate their purification and to promote endosomal escape of the toxin into recipient cells, both products were genetically engineered to express a histidine tag at the C-terminus [28]. Since SAP can inactivate prokaryotic ribosomes, their production in E. coli was induced with IPTG for only 3 h. Western blot analysis of the purified RGD-SAP, performed under reducing and non-reducing conditions, showed a single band of 30 kDa as expected for monomers (FIG. 1C), suggesting that inter-chain disulfide bonds between the ACDCRGDCFCG domains of different molecules were not formed. In contrast, two bands corresponding to monomers and dimers were detected in CYS-SAP, suggesting that an inter-chain disulfide bond was formed between the N-terminal cysteines of this protein (FIG. 1C).

RGD-SAP can Kill Integrin-Expressing Cells More Efficiently than CYS-SAP

RGD-4C can recognize αv-integrins with different affinities [29]. Integrins, such as αvβ3, αvβ5 and αvβ6, are present on a variety of tumor cells [24-26, 30]. Therefore, to identify tumor cells that could be exploited as targets to validate the targeting properties of RGD-SAP in vitro, inventors characterized the surface expression of integrins by various cancer cell lines, including U87 glioblastoma cells, RT4, RT112 and 5637 bladder cancer cells, MDA-MB-468 breast cancer cells and normal fibroblasts, by flow cytometry. The results showed that U87 cells express high levels of αvβ3, but not of αvβ5 and αvβ6, whereas RT4, RT112 and 5637 and MDA-MB-468 cells showed a moderate-high positivity for αvβ5 and αvβ6, but not of αvβ3. Normal fibroblasts expressed none of these integrins (FIG. 2A).

To assess whether the RGD domain can increase the cytotoxic effects of saporin against cancer cells, inventors then tested the cytotoxic effects of RGD-SAP and CYS-SAP on these cell lines. A stronger cytotoxic effect of RGD-SAP, compared to CYS-SAP, was observed with all cancer cells, but not with normal fibroblasts (FIG. 2B). Considering that fibroblasts do not express αvβ3 and αvβ5, i.e. integrins known to be recognized by RGD-4C, these data suggest that the stronger cytotoxic effects of RGD-SAP against cancer cells was mediated by an RGD-dependent targeting mechanism.

To verify this hypothesis, inventors performed competition experiments with the free RGD-4C peptide on 5637 cell line, selectively sensitive to RGD-SAP and representative of a human muscle-invasive model of bladder cancer. To this end, cells were treated with 0.1 μM RGD-SAP or 1 μM CYS-SAP, concentrations reflecting the different sensitivity of cells towards the two toxins, in the presence or absence of an excess of free RGD-4C. As expected, RGD-4C significantly decreased the activity of RGD-SAP, but not that of CYS-SAP (FIG. 3A). This result lends support to the hypothesis that indeed the RGD domain of RGD-SAP contribute to the cytotoxic activity of this conjugate by a receptor-mediated targeting mechanism, likely involving integrins.

It is well known that SAP induces cell apoptosis. Thus, inventors then investigated the activation of programmed cell death by analyzing caspase 3 in cells treated with RGD-SAP. To this aim, 5637 and MDA-MB-468 epithelial cells were incubated with RGD-SAP or DTT, a positive control, for 48 and 72 h. As shown in FIG. 3B, caspase 3 activation was detectable as cleaved form in all samples treated with the toxin. These and the above results indicate that fusing the RGD moiety to SAP enhances the delivery and uptake of the toxin.

RGD-SAP is Endowed with Antitumor Activity on a Subcutaneous Model of Bladder Cancer

The anti-tumor activity of RGD-SAP and CYS-SAP were then investigated using C57BL/6 mice bearing subcutaneous murine MB49 urothelial carcinoma cells. A preliminary experiment, performed in vitro, showed that RGD-SAP could kill these cells (FIG. 4A).

Thus, mice were treated, systemically, with 1 mg/kg of RGD-SAP or CYS-SAP at day 5. The treatment was repeated three times every five days (FIG. 4B). RGD-SAP could reduce tumor growth after the second administration, leading to marked reduction of the tumor volume in all mice and complete eradication in one mouse (FIGS. 4C and D and FIG. 9A). On the contrary, CYS-SAP did not show any effect on tumor growth, suggesting that the therapeutic effect of RGD-SAP crucially involved the RGD domain. However, the dose of RGD-SAP used in this experiment also caused loss of body weight at day 19 (FIG. 4D) and severe necrosis in the tails where the drug was injected.

To determine the minimal effective, non-toxic dose of RGD-SAP tumor-bearing mice were treated with 0.75, 0.5, 0.25 mg/kg of RGD-SAP at days 5, 10, 15 after tumor implantation, as described above. The doses of 0.75 and 0.5, but not 0.25 mg/kg, caused a significant delay of tumor growth, pointing to a dose-dependent effect (FIG. 5A and FIG. 9B), without causing loss of body weight (FIGS. 5B and C).

The toxicological effects RGD-SAP was further investigated. To this aim, inventors collected blood samples at the end of each experiment or before animal sacrifice and analyzed biochemical parameters of liver and kidney toxicity (albumin, alanine transaminase, aspartate transaminase for liver toxicity, and creatinine and urea for kidney toxicity). As shown in FIG. 6, RGD-SAP systemic toxicity was dose dependent and apparent only with the highest dose used (1 mg/kg). In fact, significantly higher values of AST and CHE were measured in mice treated with 1 mg/kg RGD-SAP, but not with the lower doses.

### RGD-SAP can Delay Tumor Growth in an Orthotopic Mouse Model of Syngeneic Bladder Cancer

The anti-tumor efficacy of RGD-SAP was then investigated in an orthotopic model of urothelial carcinoma. To this aim, inventors genetically engineered MB49 cells to express luciferase (MB49-luc). MB49-luc cells were then orthotopically implanted into immunocompetent C57BL/6 mice. Tumor engraftment and growth, as monitored by in vivo bioluminescence imaging, occurred in 100% of mice in 5-7 days after cells inoculation. This tumor model resembles advanced bladder cancer and is characterized by high proliferation rate accompanied by protrusion of the mass into the bladder lumen, obstruction of urethra, hematuria and necrosis in the tumor core a few days upon engraftment [27]. These features can lead to an inadequate drug delivery to the tumor mass [27]. To overcome this limitation, inventors decided to use mitomycin C (MMC) as a tool to slow down the tumor growth and delay necrosis formation, thereby allowing the toxin to reach tumor cells and exert its specific effect. MMC is one of the most widely used agents for preventing recurrence of superficial bladder cancer in clinics, usually administered intravesically after transurethral resection of cancer lesions [31-33]. Of note, MB49 bladder cancer cells were extremely sensitive to MMC (IC50˜2 g/ml) (FIG. 7A).

At the time of tumor detection (day 7 after cells infusion into the bladder) two experimental groups were treated with MMC through transurethral administration. A second dose of MMC was given after four days. In between, mice received a first dose of RGD-SAP (systemically, 0.5 mg/kg) or vehicle, which was repeated for three times (FIG. 7B). One group of mice was treated with RGD-SAP alone, as control. As previously demonstrated [34], in this model, after an initial increase, the bioluminescent signal drops in a time dependent manner, due to the reduced light emission from extended necrotic and hemorrhagic areas compared with vital tumor areas. In the control group the bioluminescent signal increased, reaching the maximum value after 11 days, and then started decreasing. Although a similar behavior was observed after treatment with MMC or RGD-SAP alone, the combination of these drugs greatly limited the increase of the signal (FIG. 7C), which implies a relevant anti-tumor effect. Accordingly, a significant effect on mice survival was observed in the group of mice treated with RGD-SAP/MMC combination (FIG. 7E,F), as 80% of mice were still alive when the experiment ended after 23 days. Histochemical analysis of the tumors, explanted after mouse scarification, showed that only one mouse treated with the combined therapy had a necrotic tumor, while all the others did not display any necrotic area, suggesting that this treatment consistently reduced the tumor growth. Furthermore, no evidence of toxicity was observed, judging from the lack of significant changes of body weight (FIG. 7D), as well as of albumin, alanine transaminase or urea levels in the bloodstream (FIG. 8).

### Discussion

The results show that the fusion of RGD-4C with SAP enables selective delivery of this toxin to tumors, thereby enhancing its antitumor activity. In particular, the results show that RGD-SAP can kill cells expressing the integrins αvβ3, αvβ5 and αvβ6 more efficiently than CYS-SAP, a control conjugate lacking the RGD-4C domain. As expected, the improved cytotoxic activity of RGD-SAP was inhibited, in vitro, by an excess of free RGD-4C peptide. Considering the known ability of RGD-4C to bind αvβ3 (affinity value: 8.3 nM), αvβ5 (46 nM), α531 (244 nM) and αvβ6 (380 nM) integrins (26), although with different affinities, and the known overexpression of these integrins in tumors, these findings suggest that integrin targeting was an important mechanism that contribute to the improved activity of RGD-SAP. Inventors tested the expression of αvβ3, αvβ5 and αvβ6, to associate the RGD-SAP cytotoxicity to the integrin expression on target tumor cells. U87, which expresses the highest amount of αvβ3, are the most sensitive to RGD-SAP, but also other cell lines, expressing αvβ5 and αvβ6 are enough sensitive to RGD-SAP, considerably more than the untargeted CYS-SAP. It is likely that not only αvβ3, but also the other abovementioned integrins can contribute to the RGD-SAP cytotoxicity. In addition, inventors can not exclude contribution of other RGD-interacting integrins to RGD-SAP cytotoxicity. To lower the risk that RGD-4C fusion with saporin could reduce or abolish the binding of the peptide to integrins, e.g. by steric hindrance, inventors have introduced a seven amino acids flexible linker. The higher activity of RGD-SAP with respect to untargeted SAP and the reduction of its cytotoxicity upon competition with RGD-4C peptide, suggests that with this linker RGD-4C preserved, at least partially if not at all, its functional properties after coupling to saporin. The results of in vivo studies in different mouse models of bladder cancer show that RGD-SAP can reduce tumor growth and significantly prolong animal survival without inducing detectable side effects. These results and the notion that RGD-4C is a compound with a proven utility as ligand for the targeted delivery of therapeutic molecules to αv integrins [16, 24, 26, 29], and that αv integrins are significantly over-expressed in bladder tumors in a stage and grade-dependent manner [35, 36], lend support to the hypothesis that this class of receptors may represent important molecular targets for toxin delivery to bladder cancer.

The approved clinical practice for the management of bladder cancer consists in transurethral resection of cancer lesions or by the removal of the entire organ (radical cystectomy), depending on the tumor grade and stage. Most of the times, a chemoprophylaxis regimen based on chemotherapeutics like platinum complexes or mitomycin C (MMC) is given either before surgery (neoadjuvant chemotherapy) or after surgery (adjuvant chemotherapy) to reduce the risk of cancer recurrence [33]. In case of advanced or metastatic bladder tumor, immune checkpoint inhibitors (anti-PDL1 antibodies) and tyrosine kinase inhibitors (specific for FGFR) represent the most effective targeted options, showing promising results in the treatment of specific subtypes. However, the clinical outcome of these treatments strictly relies on the presence of an elevated immune signature or FGFR2/3 specific mutation (typical of patients with a luminal I bladder cancer subtype) [37, 38].

To recapitulate advanced bladder cancer features, inventors have tested the pharmacological efficacy of our recombinant protein, systemically administered, using syngeneic bladder cancer mouse models. At first inventors used a subcutaneous cancer model to determine the optimal dosage and found that RGD-SAP can inhibit the tumor growth in a dose-dependent manner. In this model, RGD-SAP was significantly more active than the CYS-SAP control, the latter being almost completely inactive. This suggest that RGD-SAP can actively target the tumor environment and exclude a “passive targeting” mechanism potentially related to the enhanced permeability of tumor tissues. Then, inventors used an orthotopically implanted tumor model (MB49) to assess the therapeutic effect of RGD-SAP alone and in combination with MMC. The MB49 orthotopic model of advanced bladder cancer is characterized by a logarithmic proliferation rate of the tumor mass, leading in several days to the formation of an inner necrotic area, causing a sudden drop of luminescence signal [27, 39]. It is expected that the uptake of drugs in solid tumors is heterogeneous and the general distribution decreases with increasing tumor weight, since cells that are progressively distant to blood vessels and located in high-pressure regions constitute large areas of hypoxic, necrotic, or semi-necrotic tissue. Thus, this condition can limit an adequate penetration of drug administrated systemically, such as RGD-SAP, into the tumor mass. Interestingly, upon MMC pre-treatment, RGD-SAP reduced tumor growth compared to MMC alone, significantly increasing overall survival (80% of mice) and improving animal welfare. Notably, RGD-SAP has shown a low cytotoxic activity on MB49 cells in vitro, suggesting that its activity in vivo could be related to the targeting of microenvironment components as well. Indeed, αvβ3 is expressed by the endothelium in the neo-angiogenic blood vessels [16-19] and it represent a potential target of RGD-SAP.

Intra-tumoral heterogeneity represents a major obstacle to cancer therapeutics, including conventional chemotherapy, immunotherapy, and targeted therapies. Due to its potential effects on tumor cells and microenvironment, RGD-SAP may represent a good therapeutic tool for bladder cancer. In addition, by inhibiting proteins synthesis, SAP acts in a cell cycle independent manner, thus targeting both quiescent and rapidly dividing tumor cells. This feature makes it suitable to contrast both aggressively growing cancers and tumors with slower progression. RGD-SAP can be employed also in combination with other therapeutic options based on different mechanisms of action, e.g. inhibition of DNA synthesis, cell division, and signal transduction.

### Conclusions

The present study demonstrates that the fusion of RGD-4C to SAP enables specific delivery of the toxin to the tumor mass and enhances its anti-tumor activity in bladder cancer models without showing detectable side effects. The RGD-SAP may be potentially applicable to other solid tumors, especially in combination with other therapeutic agents to tackle tumor heterogeneity.

### EXAMPLE 2

### Materials and Methods

### Cell Cultures

Human bladder RT4, RT112, 5637 were maintained in RPMI 1640 supplemented with 10% fetal calf serum (FCS), 2 mM L-glutamine and antibiotics (100 U/mL penicillin and 100 μg/mL streptomycine-sulphate); glioblastoma U87 cancer cell lines as well as skin fibroblast cells were maintained in DMEM supplemented with 10% FCS, 2 mM L-glutamine and antibiotics (100 U/mL penicillin and 100 μg/mL streptomycine-sulphate).

Cloning of NGR-SAP, RGD-SAP, CgA-SAP and CYS-SAP in pET22b Vector

SAP fused with the sequences encoding CNGRCG (NGR motif), ACDCRGDCFCG (RGD-4C), FETLRGDERILSILRHQNLLKELQD (CgA39-63) or CGGSGG at its N-terminus were prepared by GenScript (New Jersey, USA). The nucleotide sequences were obtained from the corresponding amino acid sequences of SAP and optimized for the expression in E. coli. A GGSSRSS sequence was interposed between the targeting domains (NGR motif, RGD-4C and CgA39-63) and SAP as a spacer and a 6×His tag was added at the C-terminus to allow the purification by affinity chromatography. The whole encoding sequences were inserted into the pET22b(+) expression vector (Novagen), by using NdeI and EcoRI restriction sites. Ligation products were used to transformed the E. coli strain DH5alpha (Invitrogen).

### Expression and Purification of SAP-Based Recombinant Proteins

The expression of NGR-SAP, RGD-SAP, CgA-SAP and CYS-SAP in transformed BL21(DE3) E. coli cells (Novagen) was induced for 3 hours at 37° C. with 0.1 mM IPTG. Bacterial pellet from 1 L culture was resuspended in 15 ml of 50 mM Tris-HCl, pH 7.5, supplemented with a cocktail of protease inhibitor (Sigma-Aldrich). Soon after, 10 mM of imidazole, lysozyme (250 μg/ml) and DNAse (20 μg/ml) were added. The bacterial solution was then incubated on ice for 45 min, subjected to 3 cycles of sonication (using a UW3100 Bandelin sonicator operating at 60% power; 2 min cycle duration with 1 sec pulse and 1 sec pause), and centrifuged at 4° C. for 25 min at 10000×g. Soluble recombinant proteins contained in the supernatant were then purified by metal chelate affinity chromatography using a HisTrap HP 5 ml column (GE Healthcare Life Sciences) equilibrated in Tris-HCl 50 mM pH 7.5, 300 mM NaCl supplemented with 10 mM imidazole (and 5 mM DTT for CYS-SAP) operated with the AktaPurifier10 FPLC system. To elute proteins, imidazole concentration was increased step by step up to 500 mM in 20 column volumes (CV). The fractions containing the target proteins were dialyzed against 50 mM Tris-HCl, pH 7.5 (CYS-SAP) or 50 mM bicine, pH 8.2 (NGR-SAP, RGD-SAP and CgA-SAP) at 4° C. for 16 hours. A cation exchange chromatography on HiTrap SP Sepharose FF column (GE Healthcare Life Sciences) was then performed for further purification, using a 20 CV gradient up to 1 M NaCl for protein elution. The proteins were then concentrated using 10 kDa cutoff Amicon centrifugal filters (Millipore-Sigma) and dialyzed against PBS with slide A lyzer dialysis cassette (Thermo Fisher). All solutions used in purification steps were prepared with sterile and endotoxin-free water (S.A.L.F. Laboratorio Farmacologico SpA, Bergamo, Italy). Protein concentration was measured using the BCA Protein Assay DC™ Kit (BioRad). Protein purity and identity was checked by SDS-PAGE and Western blotting.

### Protein Electrophoresis in Polyacrylamide Gel (SDS-PAGE)

This technique allows to separate the proteins present in a sample by migrating them, in an electric field, through the meshes that are formed in the polyacrylamide gel. This migration is made possible by the presence of SDS (sodiumdodecyl sulfate), an anionic detergent capable of complexing with proteins and giving them a negative charge per unit of mass. The result is that these proteins are separated during migration according to their molecular weight. Protein samples were prepared with a specific buffer consisting of 50 mM Tris-HCl at pH 6.8, 10% glycerol, 1.6% SDS, 0.1% bromophenol with the addition of 100 nM of 2-β-mercaptoethanol for form a buffer of a reducing nature. The samples were then brought to a boil for about 5 minutes at 95° C. subsequently they were loaded into the wells of the gel and made to separate according to their molecular weight in a vertical manner. The polyacrylamide gel consists of two parts: a part called Running (lower) which is then the portion where the separation of proteins takes place and a part called Stacking (upper) in which the various wells are present. The electrophoretic run took place in a special running chamber in the presence of a buffer (1× running buffer) for about 1.5 h with an amperage of about 25 mA per gel present.

### Western Blot Analysis

Cells were washed twice with cold PBS, collected by scraping and centrifuged 5 min at 300 g. Cells were lysed for 30 min on ice in ice-cold buffer (50 mM Tris-HCl, pH 7.5, containing 150 mM NaCl, 2 mM NaF, 1 mM EDTA, 1 mM EGTA, 1 mM Na3VO4, 1 mM PMSF, 75 mU/ml aprotinin (Sigma-Aldrich), 1% TritonX-100 and a proteinase inhibitor Cocktail® (Sigma-Aldrich). Cell lysates were centrifuged at 10000×g at 4° C. for 10 min and the supernatants were recovered and quantified for total protein content. Equal amounts of cell protein extracts were separated by SDS-PAGE under reducing conditions unless stated otherwise. For western blot analysis, proteins were transferred onto a nitrocellulose membrane, incubated with 5% non-fat powdered milk in TBS-T (0.5% Tween-20) for 1 hour and then with the following antibodies: anti-saporin anti-serum (rabbit, 1:5000), anti-caspase 3 (rabbit, 1:2000, clone E87, Abcam), anti-beta actin (mouse, 1:10000, clone AC-15, Sigma-Aldrich). The antibody binding was detected using a secondary horseradish peroxidase conjugated antibodies (donkey anti-mouse/rabbit IgG HRP-linked, GE Healthcare) and an enhanced chemiluminescent (ECL, Merck Millipore).

Seed-extracted SAP used as positive control for Western blot analysis was purchased from Advanced Targeting Systems.

### Cell Viability Assay (MTT)

Cultured cell lines (5×10³ cells/well) were seeded in 96 wells plates and incubated for 72 h with various amounts of NGR-SAP, RGD-SAP, CgA-SAP, CYS-SAP, ATF-SAP or seed SAP at 37° C., 5% CO₂. Cell viability was then quantified by 3-(4,5-dimethylthiazol-2-yl)-2,5-diphenyltetrazolium bromide staining (MTT) (working solution 0.5 μg/ml). After 1 h of incubation, the supernatants were removed, the formazan crystals were dissolved with dimethyl sulfoxide and the absorbance at 570 nm was measured using a microtiter plate reader.

### Statistical Analysis

All in vitro experiments were performed at least in triplicate. Statistical significance was determined using a 2-tailed Student's t test. Tests symbols mean: *p<0.05; **p<0.01; ***p<0.001; ns, not significant.

### Results

### Production and Characterization of SAP-Based Recombinant Proteins

The coding sequences for the SAP-based chimeras were designed to insert a targeting sequence (NGR motif, RGD-4C and CgA39-63) at the N-terminus of the toxin.

The RGD-4C sequence consists of 11 aa (ACDCRGDCFCG) where the RGD motif is contained. The receptor recognized by the targeting domain is the integrin αvβ3, although other integrins (αvβ5, αvβ6 and α5p 1) have been reported to interact with RGD-4C, even if at lesser extent (Kapp et al. Sci Rep 2017).

In the CgA-SAP fusion protein, only a 25 amino acid fragment of the chromogranin A sequence (439 total amino acids), spanning from Phe 39 to Asp 63 (FETLRGDERILSILRHQNLLKELQD), was used. The CgA39-63 contains a RGD motif followed by an amphipathic α-helix, both very critical for the binding affinity to αvβ3>αvβ5>αvβ6˜αvβ8>α5β1 (Curnis et al. Cell. Mol. Life Sci. 2012).

The NGR motif consists of 6 aa (CNGRCG) and is known to bind the aminopeptidase N (APN), also called CD13, up-regulated in endothelial cells within mouse and human tumors (Pasqualini et al. Cancer Res. 2000).

A flexible linker was introduced to separate the targeting peptides from SAP in order to better expose them and favor the binding to the receptors. In parallel, a SAP variant with a Cys residue in place of the targeting domain (CYS-SAP) was produced as a control (FIGS. 1A and B).

The production of SAP-based recombinant proteins was obtained only upon IPTG induction for 3 hours, to minimize the self-intoxication, since SAP cytotoxic also for prokaryotes (FIG. 1).

Western blot analysis of the purified SAP-based recombinant proteins, performed under reducing and non-reducing conditions, showed a single band around 30 kDa as expected for monomers (FIG. 1C), excluding the formation of inter-chain disulfide bonds in the targeting domains RGD-4C and NGR motif. In contrast, two bands corresponding to monomers and dimers were detected in CYS-SAP, suggesting that an inter-chain disulfide bond was formed between the N-terminal cysteines of this protein (FIG. 1C).

### NGR-SAP, RGD-SAP and CgA-SAP Show Activity and Target Selectivity on Tumor Cells

To validate the targeting properties of SAP-based recombinant proteins, U87 cells expressing high levels of αvβ3, RT4, RT112 and 5637 cells showing a moderate to high positivity for αvβ5 and αvβ6 were used. Normal fibroblasts expressed none of these integrins were used as control. To assess whether the targeting domains can increase the cytotoxic effects of SAP against cancer cells, we tested the impact of NGR-SAP, RGD-SAP and CgA-SAP as well as CYS-SAP and a seed-derived SAP. A stronger cytotoxic effect of all SAP-based, targeted recombinant proteins compared to recombinant CYS-SAP and seed-extracted SAP was observed with all cancer cell lines, but not with normal fibroblasts (FIG. 10). Since fibroblasts do not express αvβ3, αvβ5 and αvβ6 integrins known to be recognized by RGD-4C and CgA39-63, these data suggest that the cytotoxic effects was mediated by a mechanism dependent on specific targeting domains.

### REFERENCES

1. Vago R, Ippoliti, R., Fabbrini, M. S.: Current status & Biomedical applications of Ribosome-inactivating proteins. In: Antitumor Potential and other Emerging Medicinal Properties of Natural Compounds. Edited by Ng EFFTB: Springer; 2013: 145-179.
2. Fabbrini M S, Katayama M, Nakase I, Vago R: Plant Ribosome-Inactivating Proteins: Progresses, Challenges and Biotechnological Applications (and a Few Digressions). Toxins (Basel) 2017, 9(10).
3. Musidlak O, Nawrot R, Gozdzicka-Jozefiak A: Which Plant Proteins Are Involved in Antiviral Defense? Review on In Vivo and In Vitro Activities of Selected Plant Proteins against Viruses. Int J Mol Sci 2017, 18(11).
4. Lapadula W J, Ayub M J: Ribosome Inactivating Proteins from an evolutionary perspective. Toxicon 2017, 136:6-14.
5. Rust A, Partridge L J, Davletov B, Hautbergue G M: The Use of Plant-Derived Ribosome Inactivating Proteins in Immunotoxin Development: Past, Present and Future Generations. Toxins (Basel) 2017, 9(11).
6. Giansanti F, Flavell D J, Angelucci F, Fabbrini M S, Ippoliti R: Strategies to Improve the Clinical Utility of Saporin-Based Targeted Toxins. Toxins (Basel) 2018, 10(2).
7. Fuchs H: Dianthin and Its Potential in Targeted Tumor Therapies. Toxins (Basel) 2019, 11(10).
8. Zuppone S, Fabbrini M S, Vago R: Hosts for Hostile Protein Production: The Challenge of Recombinant Immunotoxin Expression. Biomedicines 2019, 7(2).
9. Della Cristina P, Castagna M, Lombardi A, Barison E, Tagliabue G, Ceriotti A, Koutris I, Di Leandro L, Giansanti F, Vago R et al: Systematic comparison of single-chain Fv antibody-fusion toxin constructs containing Pseudomonas Exotoxin A or saporin produced in different microbial expression systems. Microb Cell Fact 2015, 14:19.
10. Dhez A C, Benedetti E, Antonosante A, Panella G, Ranieri B, Florio T M, Cristiano L, Angelucci F, Giansanti F, Di Leandro L et al: Targeted therapy of human glioblastoma via delivery of a toxin through a peptide directed to cell surface nucleolin. J Cell Physiol 2018, 233(5):4091-4105.
11. Errico Provenzano A, Posteri R, Giansanti F, Angelucci F, Flavell S U, Flavell D J, Fabbrini M S, Porro D, Ippoliti R, Ceriotti A et al: Optimization of construct design and fermentation strategy for the production of bioactive ATF-SAP, a saporin based anti-tumoral uPAR-targeted chimera. Microb Cell Fact 2016, 15(1):194.
12. Thakur M, Mergel K, Weng A, von Mallinckrodt B, Gilabert-Oriol R, Durkop H, Melzig M F, Fuchs H: Targeted tumor therapy by epidermal growth factor appended toxin and purified saponin: an evaluation of toxicity and therapeutic potential in syngeneic tumor bearing mice. Mol Oncol 2013, 7(3):475-483.
13. Zuppone S, Assalini C, Minici C, Bertagnoli S, Branduardi P, Degano M, Fabbrini M S, Montorsi F, Salonia A, Vago R: The anti-tumoral potential of the saporin-based uPAR-targeting chimera ATF-SAP. Sci Rep 2020, 10(1):2521.
14. di Leandro L, Giansanti F, Mei S, Ponziani S, Colasante M, Ardini M, Angelucci F, Pitari G, d'Angelo M, Cimini A et al: Aptamer-Driven Toxin Gene Delivery in U87 Model Glioblastoma Cells. Front Pharmacol 2021, 12:588306.
15. Fuchs H, Bachran C, Li T, Heisler I, Durkop H, Sutherland M: A cleavable molecular adapter reduces side effects and concomitantly enhances efficacy in tumor treatment by targeted toxins in mice. J Control Release 2007, 117(3):342-350.
16. Wang H, Chen K, Cai W, Li Z, He L, Kashefi A, Chen X: Integrin-targeted imaging and therapy with RGD4C-TNF fusion protein. Mol Cancer Ther 2008, 7(5):1044-1053.
17. Bianconi D, Unseld M, Prager G W: Integrins in the Spotlight of Cancer. Int J Mol Sci 2016, 17(12).
18. Blandin A F, Renner G, Lehmann M, Lelong-Rebel I, Martin S, Dontenwill M: beta1 Integrins as Therapeutic Targets to Disrupt Hallmarks of Cancer. Front Pharmacol 2015, 6:279.
19. Hynes R O: Integrins: bidirectional, allosteric signaling machines. Cell 2002, 110(6):673-687.
20. Li M, Wang W, Li M, Wu X, Setrerrahmane S, Xu H: Integrins as attractive targets for cancer therapeutics. Acta Pharmaceutica Sinica B 2021, in press.
21. Brooks P C, Stromblad S, Klemke R, Visscher D, Sarkar F H, Cheresh D A: Antiintegrin alpha v beta 3 blocks human breast cancer growth and angiogenesis in human skin. J Clin Invest 1995, 96(4):1815-1822.
22. Friedlander M, Brooks P C, Shaffer R W, Kincaid C M, Varner J A, Cheresh D A: Definition of two angiogenic pathways by distinct alpha v integrins. Science 1995, 270(5241):1500-1502.
23. Bodero L, Lopez Rivas P, Korsak B, Hechler T, Pahl A, Muller C, Arosio D, Pignataro L, Gennari C, Piarulli U: Synthesis and biological evaluation of RGD and isoDGR peptidomimetic-alpha-amanitin conjugates for tumor-targeting. Beilstein J Org Chem 2018, 14:407-415.
24. Curnis F, Gasparri A, Sacchi A, Longhi R, Corti A: Coupling tumor necrosis factor-alpha with alphaV integrin ligands improves its antineoplastic activity. Cancer Res 2004, 64(2):565-571.
25. Danhier F, Le Breton A, Preat V: RGD-based strategies to target alpha(v) beta(3) integrin in cancer therapy and diagnosis. Mol Pharm 2012, 9(11):2961-2973.
26. Temming K, Schiffelers R M, Molema G, Kok R J: RGD-based strategies for selective delivery of therapeutics and imaging agents to the tumour vasculature. Drug Resist Updat 2005, 8(6):381-402.
27. Loskog A, Ninalga C, Hedlund T, Alimohammadi M, Malmstrom P U, Totterman T H: Optimization of the MB49 mouse bladder cancer model for adenoviral gene therapy. Lab Anim 2005, 39(4):384-393.
28. Ferrer-Miralles N, Corchero J L, Kumar P, Cedano J A, Gupta K C, Villaverde A, Vazquez E: Biological activities of histidine-rich peptides; merging biotechnology and nanomedicine. Microb Cell Fact 2011, 10:101.
29. Kapp T G, Rechenmacher F, Neubauer S, Maltsev O V, Cavalcanti-Adam E A, Zarka R, Reuning U, Notni J, Wester H J, Mas-Moruno C et al: A Comprehensive Evaluation of the Activity and Selectivity Profile of Ligands for RGD-binding Integrins. Sci Rep 2017, 7:39805.
30. Curnis F, Sacchi A, Longhi R, Colombo B, Gasparri A, Corti A: IsoDGR-tagged albumin: a new alphavbeta3 selective carrier for nanodrug delivery to tumors. Small 2013, 9(5):673-678.
31. Bosschieter J, Nieuwenhuijzen J A, van Ginkel T, Vis A N, Witte B, Newling D, Beckers G M A, van Moorselaar R J A: Value of an Immediate Intravesical Instillation of Mitomycin C in Patients with Non-muscle-invasive Bladder Cancer: A Prospective Multicentre Randomised Study in 2243 patients. Eur Urol 2018, 73(2):226-232.
32. Racioppi M, Di Gianfrancesco L, Ragonese M, Palermo G, Sacco E, Bassi P: Chemoablation with Intensive Intravesical Mitomycin C Treatment: A New Approach for Non-muscle-invasive Bladder Cancer. Eur Urol Oncol 2019, 2(5):576-583.
33. Soloway M S: Expectant treatment of small, recurrent, low-grade, noninvasive tumors of the urinary bladder. Urol Oncol 2006, 24(1):58-61.
34. Jurczok A, Fornara P, Soling A: Bioluminescence imaging to monitor bladder cancer cell adhesion in vivo: a new approach to optimize a syngeneic, orthotopic, murine bladder cancer model. BJU Int 2008, 101(1):120-124.
35. Sachs M D, Rauen K A, Ramamurthy M, Dodson J L, De Marzo A M, Putzi M J, Schoenberg M P, Rodriguez R: Integrin alpha(v) and coxsackie adenovirus receptor expression in clinical bladder cancer. Urology 2002, 60(3):531-536.
36. van der Horst G, Bos L, van der Mark M, Cheung H, Heckmann B, Clement-Lacroix P, Lorenzon G, Pelger R C, Bevers R F, van der Pluijm G: Targeting of alpha-v integrins reduces malignancy of bladder carcinoma. PLoS One 2014, 9(9):e108464.
37. Li H, Zhang Q, Shuman L, Kaag M, Raman J D, Merrill S, DeGraff D J, Warrick J I, Chen G: Evaluation of PD-L1 and other immune markers in bladder urothelial carcinoma stratified by histologic variants and molecular subtypes. Sci Rep 2020, 10(1):1439.
38. Loriot Y, Necchi A, Park S H, Garcia-Donas J, Huddart R, Burgess E, Fleming M, Rezazadeh A, Mellado B, Varlamov S et al: Erdafitinib in Locally Advanced or Metastatic Urothelial Carcinoma. N Engl J Med 2019, 381(4):338-348.
39. Mangsbo S M, Ninalga C, Essand M, Loskog A, Totterman T H: CpG therapy is superior to BCG in an orthotopic bladder cancer model and generates CD4+ T-cell immunity. J Immunother 2008, 31(1):34-42.

1. A fusion protein or conjugate comprising:
a) at least one peptide recognizing tumor endothelial cell markers, said peptide being preferably:
an αv-integrin ligand, preferably a ligand of αvβ3, αvβ5, αvβ8, α501 and/or αvβ6 integrin, more preferably said peptide comprising a sequence comprising a RGD motif or of a CgA sequence
or
a ligand of CD13 receptor, more preferably said ligand comprising a sequence comprising a NGR motif or functional fragments or derivatives or a biologically active variant thereof and
b) saporin or functional fragments, derivatives or a biologically active variant thereof.

2. The fusion protein or conjugate of claim 1 being able to target tumor endothelial cell markers.

3. The fusion protein or conjugate of claim 1, wherein the saporin has at least 80% sequence identity with, or comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK)
and/or wherein:
i. the sequence comprising a RGD motif has at least 80% sequence identity with, or comprises SEQ ID NO: 2 (ACDCRGDCFCG), preferably it comprises SEQ ID NO:2 or
ii. the sequence comprising a RGD motif or the CgA sequence is CgA39-63, preferably it has at least 80% sequence identity with, or comprises SEQ ID NO: 3 (FETLRGDERILSILRHQNLLKELQD), preferably it comprises SEQ ID NO: 3 or
iii. the sequence comprising a NGR motif has at least 80% sequence identity with, or comprises SEQ ID NO: 4 (CNGRCG), preferably it comprises SEQ ID NO: 4, and/or wherein the fusion protein or conjugate further comprises a linker component between a) and b) of claim 1, said linker preferably comprises at least 3 amino acids, it preferably has at least 90% sequence identity with SEQ ID NO: 5 (GGSSRSS), more preferably it comprises SEQ ID NO: 5 (GGSSRSS)
and/or wherein the fusion protein or conjugate comprises from N-terminus to C-terminus a) and b) of claim 1
and/or wherein the fusion protein or conjugate further comprises a spacer at the C-terminus, preferably the sequence VDK, and/or an histidine tag, preferably comprising 6 histidines, at the C-terminus.

4. The fusion protein or conjugate according to claim 1, comprising from N-terminus to C-terminus:
i. a peptide recognizing tumor endothelial cell markers comprising a sequence comprising a RGD motif having at least 80% sequence identity with, or comprising SEQ ID NO: 2 (ACDCRGDCFCG) or functional fragments or derivatives or a biologically active variant thereof and
ii. saporin or functional fragments, derivatives or a biologically active variant thereof.

5. The fusion protein or conjugate of claim 4, wherein the saporin has at least 80% sequence identity with, or comprises SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK).

6. The fusion protein or conjugate of claim 4 being able to target tumor endothelial cell markers and wherein:
the sequence comprising a RGD motif having at least 80% sequence identity with SEQ ID NO: 2 (ACDCRGDCFCG) or the functional fragments or derivatives or a biologically active variant thereof present the same activity of SEQ ID NO:2 and
the saporin having at least 80% sequence identity with SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK) or functional fragments, derivatives or a biologically active variant thereof present the same activity of saporin.

7. The fusion protein or conjugate according to claim 5, wherein a sequence comprising a RGD motif comprises or consists of SEQ ID NO:2.

8. The fusion protein or conjugate according to claim 4, wherein the sequence comprising a RGD motif comprises SEQ ID NO:2 or functional fragments, derivatives or a biologically active variant thereof and the saporin comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK) or functional fragments, derivatives or a biologically active variant thereof.

9. The fusion protein or conjugate according to claim 4, wherein the sequence comprising a RGD motif comprises or consists of SEQ ID NO:2 and the saporin comprises or consists of SEQ ID NO: 1 (VTSITLDLVNPTAGQYSSFVDKIRNNVKDPNLKYGGTDIAVIGPPSKEKFLRINFQSSRG TVSLGLKRDNLYVVAYLAMDNTNVNRAYYFKSEITSAELTALFPEATTANQKALEYTE DYQSIEKNAQITQGDKSRKELGLGIDLLLTFMEAVNKKARVVKNEARFLLIAIQMTAEV ARFRYIQNLVTKNFPNKFDSDNKVIQFEVSWRKISTAIYGDAKNGVFNKDYDFGFGKVR QVKDLQMGLLMYLGKPK).

10. The fusion protein or conjugate of claim 4 further comprising a linker component between the peptide recognizing tumor endothelial cell markers and saporin, wherein the linker preferably comprises at least 3 amino acids.

11. The fusion protein or conjugate of claim 10 wherein the linker has at least 90% sequence identity with SEQ ID NO: 5 (GGSSRSS), more preferably it comprises or consists of SEQ ID NO: 5 (GGSSRSS).

12. The fusion protein or conjugate of claim 1 having at least 90% sequence identity with, or comprising or consisting of
SEQ ID NO: 6
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDKHHHHHH)
or
SEQ ID NO: 23
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDK)
or
SEQ ID NO: 24
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPK)
or
SEQ ID NO: 7
(CNGRCGGGSSRSSVTSITLDLVNPTAGQYSSFVDKIRNNVKDPNLKYGGTDIAVIGPPSK
EKFLRINFQSSRGTVSLGLKRDNLYVVAYLAMDNTNVNRAYYFKSEITSAELTALFPEA
TTANQKALEYTEDYQSIEKNAQITQGDKSRKELGLGIDLLLTFMEAVNKKARVVKNEA
RFLLIAIQMTAEVARFRYIQNLVTKNFPNKFDSDNKVIQFEVSWRKISTAIYGDAKNGVF
NKDYDFGFGKVRQVKDLQMGLLMYLGKPKVDKHHHHHH)
or
SEQ ID NO: 25
(CNGRCGGGSSRSSVTSITLDLVNPTAGQYSSFVDKIRNNVKDPNLKYGGTDIAVIGPPSK
EKFLRINFQSSRGTVSLGLKRDNLYVVAYLAMDNTNVNRAYYFKSEITSAELTALFPEA
TTANQKALEYTEDYQSIEKNAQITQGDKSRKELGLGIDLLLTFMEAVNKKARVVKNEA
RFLLIAIQMTAEVARFRYIQNLVTKNFPNKFDSDNKVIQFEVSWRKISTAIYGDAKNGVF
NKDYDFGFGKVRQVKDLQMGLLMYLGKPKVDK)
or
SEQ ID NO: 26
(CNGRCGGGSSRSSVTSITLDLVNPTAGQYSSFVDKIRNNVKDPNLKYGGTDIAVIGPPSK
EKFLRINFQSSRGTVSLGLKRDNLYVVAYLAMDNTNVNRAYYFKSEITSAELTALFPEA
TTANQKALEYTEDYQSIEKNAQITQGDKSRKELGLGIDLLLTFMEAVNKKARVVKNEA
RFLLIAIQMTAEVARFRYIQNLVTKNFPNKFDSDNKVIQFEVSWRKISTAIYGDAKNGVF
NKDYDFGFGKVRQVKDLQMGLLMYLGKPK)
or
SEQ ID NO: 8
(FETLRGDERILSILRHQNLLKELQDGGSSRSSVTSITLDLVNPTAGQYSSFVDKIRNNVK
DPNLKYGGTDIAVIGPPSKEKFLRINFQSSRGTVSLGLKRDNLYVVAYLAMDNTNVNRA
YYFKSEITSAELTALFPEATTANQKALEYTEDYQSIEKNAQITQGDKSRKELGLGIDLLLT
FMEAVNKKARVVKNEARFLLIAIQMTAEVARFRYIQNLVTKNFPNKFDSDNKVIQFEVS
WRKISTAIYGDAKNGVFNKDYDFGFGKVRQVKDLQMGLLMYLGKPKVDKHHHHHH)
or
SEQ ID NO: 27
(FETLRGDERILSILRHQNLLKELQDGGSSRSSVTSITLDLVNPTAGQYSSFVDKIRNNVK
DPNLKYGGTDIAVIGPPSKEKFLRINFQSSRGTVSLGLKRDNLYVVAYLAMDNTNVNRA
YYFKSEITSAELTALFPEATTANQKALEYTEDYQSIEKNAQITQGDKSRKELGLGIDLLLT
FMEAVNKKARVVKNEARFLLIAIQMTAEVARFRYIQNLVTKNFPNKFDSDNKVIQFEVS
WRKISTAIYGDAKNGVFNKDYDFGFGKVRQVKDLQMGLLMYLGKPKVDK)
or
SEQ ID NO: 28
FETLRGDERILSILRHQNLLKELQDGGSSRSSVTSITLDLVNPTAGQYSSFVDKIRNNVK
DPNLKYGGTDIAVIGPPSKEKFLRINFQSSRGTVSLGLKRDNLYVVAYLAMDNTNVNRA
YYFKSEITSAELTALFPEATTANQKALEYTEDYQSIEKNAQITQGDKSRKELGLGIDLLLT
FMEAVNKKARVVKNEARFLLIAIQMTAEVARFRYIQNLVTKNFPNKFDSDNKVIQFEVS
WRKISTAIYGDAKNGVFNKDYDFGFGKVRQVKDLQMGLLMYLGKPK),
or functional fragments, equivalents, variants, mutants, derivatives, synthetics or recombinants functional analogues thereof.

13. The fusion protein or conjugate of claim 1 comprising or consisting of:
SEQ ID NO: 6
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDKHHHHHH)
or
SEQ ID NO: 23
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDK)
or
SEQ ID NO: 24
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPK)
or functional fragments, equivalents, variants, mutants, derivatives, synthetics or recombinants functional analogues thereof.

14. The fusion protein or conjugate of claim 1 comprising or consisting of:
SEQ ID NO: 6
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDKHHHHHH)
or
SEQ ID NO: 23
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPKVDK)
or
SEQ ID NO: 24
(ACDCRGDCFCGGGSSRSSVTSITLDLVNPTAGQYSSFVDKIRNNVKDPNLKYGGTDIAV
IGPPSKEKFLRINFQSSRGTVSLGLKRDNLYVVAYLAMDNTNVNRAYYFKSEITSAELTA
LFPEATTANQKALEYTEDYQSIEKNAQITQGDKSRKELGLGIDLLLTFMEAVNKKARVV
KNEARFLLIAIQMTAEVARFRYIQNLVTKNFPNKFDSDNKVIQFEVSWRKISTAIYGDAK
NGVFNKDYDFGFGKVRQVKDLQMGLLMYLGKPK).

15. An isolated nucleic acid encoding the fusion protein or conjugate of claim 1.

16. A recombinant expression vector comprising the isolated nucleic acid of claim 15 operably linked to regulatory sequences capable of directing expression of a gene encoding said fusion protein in a host.

17. A host cell comprising and/or expressing the fusion protein or conjugate of claim 1, said host cell preferably being selected from the group consisting of:
fungal cells, bacterial cells, preferably gram-positive Bacilli such as B. subtilis, B. licheniformis, B. megaterium, B. amyloliquefaciens, B. pumilus, gram negative bacteria such as Escherichia coli, plant protoplasts, unicellular algae, actinomycetales such as Streptomyces sp., and yeasts, such as Saccharomyces cerevisiae, Pichia pastoris, Yarrowia lipolytica, most preferably Trichoderma reesei or Bacillus.

18. A method of producing the fusion protein or conjugate of claim 1 comprising the steps of transforming a host cell with an expression vector encoding said fusion protein, culturing said host cell under conditions enabling expression of said fusion protein, and optionally recovering and purifying said fusion protein.

19. A composition comprising the fusion protein or conjugate of claim 1 and at least one pharmaceutically acceptable carrier, preferably further comprising an anti-cancer agent.

20. A method for the treatment of cancer, preferably vascularized solid tumors or irrorated solid tumors, preferably bladder cancer, lung cancer, breast cancer, colorectal cancer, ovarian cancer prostate cancer or pancreatic cancer, comprising administering the fusion protein or conjugate of claim 1 to a patient in need thereof.
